(19) United States
(12) Patent Application Publication
(10) Pub. No.: US 2016/0121023 A1
(43) Pub. Date: May 5, 2016

(54) Materials and Methods for Rescue of Ischemic Tissue and Regeneration of Tissue Integrity During Resection, Engraftment and Transplantation

(71) Applicant: Massachusetts Institute of Technology, Cambridge, MA (US)

(72) Inventors: Elazer Reuven Edelman, Brookline, MA (US); Pedro Melgar Lesmes, Cambridge, MA (US)

(21) Appl. No.: 14/926,757

(22) Filed: Oct. 29, 2015

Related U.S. Application Data
(60) Provisional application No. 62/072,750, filed on Oct. 30, 2014.

(51) Int. Cl. A61L 27/38; A61L 27/56; A61L 27/22

(52) U.S. Cl. CPC A61L 27/3808; A61L 27/222; A61L 27/56; A61L 2430/28

(57) ABSTRACT

Cell-containing implants populated with endothelial cells rescue liver donor and recipient endothelium and parenchyma from ischemic injury after major hepatectomy and engraftment. The inventions disclosed herein highlight the discovery that endothelial-hepatocyte physiologically communicate and cooperate during hepatic repair. The present inventions provide materials and methods for a new approach to improve transplant and regenerative medicine outcomes, for example, liver transplantation.

### RELATED APPLICATIONS

This application claims priority to and the benefit of provisional application Ser. No. 62/072,750, filed on Oct. 30, 2014, which is herein incorporated in its entirety by reference.

### FIELD OF THE INVENTION

The inventions disclosed herein are directed to materials and methods for treatment and rescue of ischemic tissue using cell-containing implantable materials. The inventions disclosed herein are further directed to materials and methods for the rescue and regeneration of tissue integrity during resection, engraftment and transplantation. The liver is a species of tissue suitable for use with the materials and methods of the present inventions.

### BACKGROUND

Tissue and organ failure account for many deaths world-wide. Diseases of the organs and tissues of the gastrointestinal system as well as liver, lung, kidney and thyroid tissues are typical of the clinical problem.

For example, liver disease is one of the leading causes of death in the world.¹ Hepatectomy and liver transplantation are the standard of care in patients with tumors of hepatic origin and end-stage liver disease.² Yet, in 2014, only 40% of eligible patients received a liver transplant, which translates into a shortage of about 10,000 donors per year.³ During the same period, 23% of patients from the waiting list died and an additional 20% of patients were removed from that list as they became too sick to undergo surgery.³ Recent efforts have been devoted to generate hepatocyte-like cells and organ buds for transplantation.⁴⁻⁵ However those promising tools are still far from replacing liver transplantation in clinics. Ischemic injury promotes a cascade of cellular responses that lead to inflammation, cell death, and ultimately hepatic and even multiorgan failure in recipients as well as donors.⁶⁻¹⁰ Further elucidation of the governing biology may eventually help explain these events, provide potential means of avoiding them and perhaps even increase the number and size of successful donor grafts.¹¹⁻¹⁴

Growing evidence suggests that liver sinusoidal endothelial cells (LSEC) are synergistic with hepatocyte proliferation and in establishing allograft tolerance.¹⁵⁻¹⁶ LSEC play critical protective roles controlling vascular tone, homeostasis, inflammation, and toxicant clearance.¹⁵ Preservation of a healthy LSEC phenotype is indispensable to minimization of liver injury and improvement of successful engraftment after hepatectomy and transplantation.¹⁷ Direct injection or transplantation of isolated endothelial cells have been proposed to repair organ damage or replace deficient functions,¹⁸⁻¹⁹ but the immune reaction that they engender limits meaningful clinical utility.

A technique called matrix-embedded endothelial cells (MEECs; also referred to herein as the implantable material or cell-containing implantable material) places endothelial cells in a three-dimensional biocompatible substrate, e.g., a collagen-based scaffold, that eliminates their immunogenicity in vitro and in vivo.²⁰⁻²¹, stimulates Th2 lymphocyte and M2 macrophage phenotype, and results in a muted expression pattern of adhesion molecules and chemokines and a markedly decreased expression of major histocompatibility complex (MHC) class II molecules.²²⁻²³

It is the object of the present invention to demonstrate that MEECs, when implanted in a recipient in need thereof, are a therapeutic tool for mitigating the risks of ischemia and graft rejection when used contemporaneously with tissue resection, tissue engraftment, tissue transplantation and organ transplantation, thereby maintaining tissue integrity and modulating disease.

### SUMMARY OF INVENTION

As demonstrated herein, Applicants provide materials and methods for rescue of ischemic tissue and regeneration of tissue integrity during resection, engraftment and transplantation. While Applicants' materials and methods are applicable to a variety of tissues, organs and disease states, Applicants chose to demonstrate the benefits of implantable MEECs using a hepatectomy model. It was heretofore unappreciated that MEEC implants can facilitate the recovery of hepatocyte function by protecting host endothelium from inflammation and by promoting angiogenesis after hepatectomy and liver engraftment, and examined these effects in a murine model of hepatectomy and liver engraftment.

Typically, liver transplantation is complicated by ischemic injury which promotes endothelial cell and hepatocyte dysfunction and eventually organ failure. In short, Applicants developed the following study design exemplified elsewhere herein to solve this problem. Matrix-embedded endothelial cells (MEECs) or control acellular matrices were implanted in direct contact with the remaining median lobe of donor mice undergoing partial hepatectomy (70%), or in the interface between the remaining median lobe and an autograft or allograft from the left lobe in hepatectomized recipient mice. Hepatic vascular architecture, DNA fragmentation and apoptosis in the median lobe and grafts, serum markers of liver damage and phenotype of macrophage and lymphocyte subsets in the liver after engraftment were analyzed 7 days post-op.

Applicants discovered that MEECs create a functional vascular splice in donor and recipient liver after 70% hepatectomy in mouse protecting these livers from ischemic injury, hepatic congestion and inflammation. Macrophages recruited adjacent to the vascular nodes into the implants switched to an anti-inflammatory and regenerative profile M2. MEECs improved liver function and the rate of liver regeneration and prevented apoptosis in donor liver lobes, autologous grafts, and allogeneic engraftment. Thus, MEEC implants can rescue liver donor and recipient endothelium and parenchyma from ischemic injury after major hepatectomy and engraftment. This study highlights endothelial-hepatocyte crosstalk in hepatic repair and provides a heretofore unrecognized approach to improve transplant and regenerative medicine outcomes, for example, liver transplantation.

In one aspect, the present invention is directed to a method of treating ischemic tissue comprising the steps of: contacting a surface of ischemic tissue with a composition comprising a biocompatible substrate, and endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and, incubating the combination of ischemic tissue and composition for a period of time sufficient to reduce or eliminate ischemia in the treated tissue. In one embodiment, the present invention is directed to a method wherein the composition comprises endothelial cells, when contacted with a surface of the ischemic tissue, which are greater than 80% viable and are in a quiescent phase of growth. In another embodiment, the present invention is directed to a method wherein the composition comprises endothelial cells, when contacted with a surface of the ischemic tissue, which express an immunomodulatory phenotype.

In another aspect, the present invention is directed to a method of treating resected tissue comprising the steps of: contacting a surface of resected tissue with a composition comprising a biocompatible substrate, and endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and, incubating the combination of ischemic tissue and composition for a period of time sufficient to promote viability of the resected tissue. In one embodiment, the invention is directed to a method wherein the composition comprises endothelial cells, when contacted with a surface of the ischemic tissue, which are greater than 85% viable and are in a quiescent phase of growth. In another embodiment, the invention is directed to a method wherein the composition comprises endothelial cells, when contacted with a surface of the ischemic tissue, which express an immunomodulatory phenotype.

In another aspect, the present invention is directed to a method of regenerating a tissue comprising the steps of: contacting a surface of a tissue with a composition comprising a biocompatible substrate, and endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and, incubating the combination of tissue and composition for a period of time sufficient to promote viability and regeneration of the tissue. In one embodiment, the method is directed to a method wherein the regenerating tissue is an ischemic tissue, a resected tissue or a transplanted tissue. In another embodiment, the method is directed to a method wherein the composition comprises endothelial cells, when contacted with a surface of the ischemic tissue, which are greater than 85% viable and are in a quiescent phase of growth. In yet another embodiment, the invention is directed to a method wherein the composition comprises endothelial cells, when contacted with a surface of the ischemic tissue, which express an immunomodulatory phenotype.

In another aspect, the present invention is directed to a method of grafting a donor tissue with a host tissue comprising the steps of: contacting a surface of a host tissue and a donor tissue with a composition comprising a biocompatible substrate, and endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and, incubating the combination of host tissue, donor tissue and composition for a period of time sufficient to promote formation of a graft comprising host tissue, donor tissue and the composition wherein the composition provides a vascular bridge comprising tubular structures which connect the donor tissue to the host tissue thereby facilitating graft formation.

In another aspect, the present invention is directed to a method of tissue transplantation comprising the steps of: contacting a surface of a donor tissue with a composition comprising a biocompatible substrate, and endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and, incubating the combination of donor tissue and composition within a transplant recipient for a period of time sufficient to promote viability and integration of the transplanted tissue.

In another aspect, the present invention is directed to a method of forming anastomoses comprising the steps of: contacting a surface of each of two tissues with a composition comprising a biocompatible substrate, and endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and, incubating the combination of two tissues and composition for a period of time sufficient to promote formation of an anastomoses wherein the composition promotes formation of a vascular bridge comprising tubular structures which connect the tissues thereby facilitating anastomoses formation.

In another aspect, the present invention is directed to a method of inducing de novo formation of vascular structures comprising the steps of: contacting a surface of a tissue with a composition comprising a biocompatible substrate, and endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and, incubating the combination of tissue and composition for a period of time sufficient to promote formation of de novo formation of vasculature within the composition wherein the composition promotes formation of vascularized anatomical structures which are tubular and support blood flow.

In another aspect, the present invention is directed to an implantable composition comprising: a tissue or segment thereof, and a cell-containing composition comprising a biocompatible substrate, and endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells. In one embodiment, the tissue or segment thereof is in contact with the cell-containing composition.

In another aspect, the present invention is directed to a tissue preparation suitable for transplantation comprising: an organ or a segment thereof, and a cell-containing composition comprising a biocompatible substrate, and endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells. In one embodiment, the organ or segment thereof is in contact with the cell-containing composition.

### FIGURES AND FIGURE LEGENDS

The patent or application file contains at least one drawing executed in color. Copies of this patent or patent application publication with color drawing(s) will be provided by the Office upon request and payment of the necessary fee.

FIG. 1(A-I). Beneficial effects of MEECs prevent liver damage in ischemic median lobe after 70% hepatectomy. C57BL/6 mice underwent 70% hepatectomy (excision of left lobe and half of median lobe):

FIG. 1(A) Macroscopic aspect of a pre-op median lobe.

FIG. 1(B) Macroscopic aspect of a median lobe 7 days post-op.

FIG. 1(C) Macroscopic aspect of a median lobe with acellular denatured collagen implants (Gel control) 7 days post-op.

FIG. 1(D) Macroscopic aspect of a median lobe treated with matrix-embedded endothelial cells (MEECs) 7 days post-op.

FIG. 1(E) Analysis of vascularity in whole liver by angiography (intracardiac perfusion of FITC-dextran, MW 2×10⁶ Da) using intravital multiphoton microscopy. Macrophages were stained in red. Representative images of the vascular network at the interface between the remaining median lobe and denatured collagen or MEECs are shown in green; macrophages are shown in red and intravascular merge of angiography and Texas red-dextran is shown in yellow.

FIG. 1(F) Representative images of angiography and quantitative analysis of vascular diameter (congestion) and functional number of vessel branches in the hepatic median lobe of sham or hepatectomized mice (HP) in the presence or absence of acellular control implants (HP+Gel) or MEECs (HP+MEECs) 7 days post-op.

FIG. 1(G) Gene expression of hepatocyte growth factor (HGF) in ischemic median lobe assessed by Real-time PCR.

FIG. 1(H) The terminal deoxynucleotidyl transferase dUTP nick-end labeling (TUNEL) assay was used in median liver lobes from hepatectomized mice in contact with acellular implants or MEECs to detect cell death. Representative images of apoptotic nuclei are shown in green. Nuclei were stained with DAPI in blue. Quantification of cell death is described elsewhere herein. Data are represented as mean±s.e.m. **P<0.01, ***P<0.001, analysis of variance (ANOVA) or t-student when appropriate.

FIG. 1(I) Assessment of apoptosis in median liver lobes from hepatectomized mice in contact with acellular implants or MEECs suing Western blot corresponding to active caspase 3. Representative images of three samples of each group to detect active caspase 3 and the housekeeping β-actin are plotted. Scale bars, 100 μm. Data are represented as mean±s.e.m. **P<0.01, ***P<0.001, analysis of variance (ANOVA) or t-student when appropriate.

FIG. 2(A-D). Vascular and immunomodulatory effects of MEECs in contact with ischemic median lobe improve liver regeneration and function. C57BL/6 mice underwent 70% hepatectomy (excision of left lobe and half of median lobe):

FIG. 2(A) Representative images of angiography and quantitative analysis of vascular diameter (congestion) and angiogenesis (number of anastomoses) in the hepatic right lobe of sham or hepatectomized mice (HP) in the presence or absence of acellular implants (HP+Gel) or MEECs (HP+MEECs) 7 days post-op.

FIG. 2(B) Representative images and quantitative analysis of total number of macrophages and contacts with vessels in the hepatic right lobe analyzed by injection of 70 kDa Texas red-dextran 2 hours before sacrifice and angiography (intracardiac perfusion of FITC-dextran, MW 2×10⁶ Da) using intravital multiphoton microscopy. Macrophages are shown in red and intravascular merge of angiography and Texas red-dextran is shown in yellow.

FIG. 2(C) Quantification of serum markers of liver damage including Alanine Aminotransferase (ALT) and Aspartate Aminotransferase (AST) in hepatectomized mice in the presence of acellular implants or MEECs. Data are represented as mean±s.e.m. *P<0.05, **P<0.01, ***P<0.001, analysis of variance (ANOVA) or t-student when appropriate.

FIG. 2(D) Assessment of liver restoration rate in sham or hepatectomized mice in the presence or absence of acellular implants or MEECs. Liver restoration rate was calculated as liver weight/body weight×100. Scale bars, 100 μm. Data are represented as mean±s.e.m. *P<0.05, **P<0.01, ***P<0.001, analysis of variance (ANOVA) or t-student when appropriate.

FIG. 3(A-D). Hepatic immunomodulation of gene expression profiles of macrophages and T helper lymphocytes after implantation of MEECs:

FIG. 3(A) Quantification of M1 (iNOS, COX-2 and IL1-β) gene expression profiles by Real-time PCR in sham or hepatectomized mice in the presence or absence of acellular implants (Gel) or MEECs. Data are represented as mean of fold change±s.e.m. *P<0.05, **P<0.01, ***P<0.001, analysis of variance (ANOVA).

FIG. 3(B) Quantification of M2 (Arg1, MRC1 and Retn1a) gene expression profiles by Real-time PCR in sham or hepatectomized mice in the presence or absence of acellular implants (Gel) or MEECs. Data are represented as mean of fold change±s.e.m. *P<0.05, **P<0.01, ***P<0.001, analysis of variance (ANOVA).

FIG. 3(C) Quantification of gene expression profiles of Th1 (INFγ and IL-2) by Real-time PCR in sham or hepatectomized mice in the presence or absence of acellular implants (Gel) or MEECs. Data are represented as mean of fold change±s.e.m. *P<0.05, **P<0.01, ***P<0.001, analysis of variance (ANOVA).

FIG. 3(D) Quantification of gene expression propfiles of Th2 (IL-4 and IL-10) by Real-time PCR in sham or hepatectomized mice in the presence or absence of acellular implants (Gel) or MEECs. Data are represented as mean of fold change±s.e.m. *P<0.05, **P<0.01, ***P<0.001, analysis of variance (ANOVA).

FIG. 4(A-H). Beneficial effects of MEECs prevent liver damage after autologous engraftment:

FIG. 4(A) Schematic representation of surgical implantation of MEECs or acellular implants in the interface between the ischemic median liver lobe and the donated graft from the left liver lobe.

FIG. 4 (B) Macroscopic aspect of median lobe and autologous grafts implanted with acellular denatured collagen or

FIG. 4(C) Macroscopic aspect of median lobe and autologous grafts implanted with MEECs 7 days post-op.

FIG. 4(D) Analysis of vascularity in the interface between median liver lobe and autologous graft by angiography using intravital multiphoton microscopy. Representative images of the vascular network at the interface between the remaining median lobe, acellular denatured collagen or MEECs and the graft are shown in green. Vascularization of implants of MEECs in contact with ischemic liver lobe. Generated functional blood vessels into implants of MEECs were visualized by angiography (intracardiac perfusion of FITC-dextran, MW 2×106 Da) using intravital multiphoton microscopy. Functional blood vessels are shown in green. Magnification 20×.

FIG. 4(E) Representative images of angiography and quantitative analysis of vascular diameter (congestion) and functional number of vessel branches in the hepatic median lobe of hepatectomized mice in the presence of acellular implants (HP+Gel) or MEECs (HP+MEECs) 7 days post-op.

FIG. 4(F) TUNEL assay was performed to detect intragraft cell death in autologous liver grafts in contact with acellular implants or MEECs. Representative images of apoptotic nuclei are shown in green. Nuclei were stained with DAPI in blue. Quantification of cell death is shown elsewhere herein.

FIG. 4(G) Assessment of apoptosis using Western blot corresponding to active caspase 3 was performed in autologous liver grafts from mice in contact with acellular implants or MEECs. Representative images of three samples of each group to detect active caspase 3 and the housekeeping β-actin are plotted.

FIG. 4(H) Quantification of serum markers of liver damage Alanine Aminotransferase (ALT) and Aspartate Aminotransferase (AST) in hepatectomized mice in the presence of acellular implants or MEECs. Scale bars, 100 μm. Data are represented as mean±s.e.m. *P<0.05, **P<0.01, ***P<0.001, analysis of variance (ANOVA) or t-student when appropriate.

FIG. 5(A-H). Beneficial effects of MEECs prevent liver damage after allogeneic engraftment:

FIG. 5(A) Macroscopic aspect of median lobe and allogeneic grafts implanted with acellular denatured collagen (Gel).

FIG. 5(B) Macroscopic aspect of median lobe and allogeneic grafts implanted with MEECs 7 days post-op.

FIG. 5(C) Analysis of vascularity in the interface between median liver lobe and allogeneic graft by angiography using intravital multiphoton microscopy. Representative images of the vascular network at the interface between the remaining median lobe, acellular Denatured collagen or MEECs and the graft are shown in green.

FIG. 5(D) Representative images of angiography and quantitative analysis of vascular diameter (congestion) and functional number of vessel branches in the hepatic median lobe of hepatectomized mice in the presence of acellular implants (HP+Gel) or MEECs (HP+MEECs) 7 days post-op.

FIG. 5(E) TUNEL assay was performed in allogeneic liver grafts in contact with acellular implants or MEECs to detect intragraft cell death. Representative images of apoptotic nuclei are shown in green. Nuclei were stained with DAPI in blue. Quantification of cell death is shown below.

FIG. 5(F) Assessment of apoptosis performed by Western blot corresponding to active caspase 3 in allogeneic liver grafts from mice in contact with acellular implants or MEECs. Representative images of three samples of each group to detect active caspase 3 and the housekeeping β-actin are plotted.

FIG. 5(G) Intragraft gene expression profile of immunotolerance expressed as Th1 (INFγ and IL-2) and Th2 (IL-4 and IL-10) cytokine expression analyzed by Real-Time PCR.

FIG. 5(H) Quantification of serum markers of liver damage Alanine Aminotransferase (ALT) and Aspartate Aminotransferase (AST) in mice with allografts in the presence of acellular implants or MEECs. Scale bars, 100 μm. Data are represented as mean±s.e.m. *P<0.05, **P<0.01, ***P<0.001, analysis of variance (ANOVA) or t-student when appropriate.

FIG. 6 Vascularization of implants of MEECs in contact with ischemic liver lobe. Generated functional blood vessels into implants of MEECs were visualized by angiography (intracardiac perfusion of FITC-dextran, MW 2×106 Da) using intravital multiphoton microscopy. Functional blood vessels are shown in green. Magnification 20×.

FIG. 7 Source of angiogenesis into implants of MEECs. HUVECs constitutively expressing GFP were seeded in gelfoams. Vascularity was analyzed in the interface between implants of HUVECs expressing GFP and implanted median liver lobes of hepatectomized mice by angiography (intracardiac perfusion of Texas red-dextran 70 kda using intravital multiphoton microscopy. Representative image of new vascular anastomoses in the implant interface coming from the extension of hepatic vessels (in red) and from MEEC-generated vessels (in yellow) are shown in the left panel. Control of expression of GFP in HUVEC-GFP cells is shown in the middle panel. Negative control using non-GFP HUVECs is shown in the right panel.

FIG. 8A Quantification of HGF gene expression profile by Real-time PCR in hepatectomized animals receiving autologous grafts in the presence or absence of acellular implants (Gel) or MEECs.

FIG. 8B Quantification of HGF gene expression profile by Real-time PCR in hepatectomized animals receiving allogeneic grafts in the presence or absence of acellular implants (Gel) or MEECs.

FIG. 9A Beneficial effects of MEECs preventing liver damage in ischemic median lobe after autologous and allogeneic engraftment. TUNEL assay was performed to detect cell death in median liver lobe after autologous engraftment in contact with acellular implants or MEECs. Representative images of apoptotic nuclei are shown in green. Nuclei were stained with DAPI in blue. Quantification of cell death is shown below.

FIG. 9B Beneficial effects of MEECs preventing liver damage in ischemic median lobe after autologous and allogeneic engraftment. TUNEL assay was performed in median liver lobe after allogeneic engraftment. Representative images of apoptotic nuclei are shown in green. Nuclei were stained with DAPI in blue. Quantification of cell death is shown below.

### DETAILED DESCRIPTION

Applicants' invention is based on an appreciation that ischemic injury promotes endothelial dysfunction in recipient livers and grafts during liver transplantation. Liver endothelial cell dysfunction or a failure of mobilization of endothelial progenitors impair liver regeneration. Recovery of blood perfusion and hepatic mass is critical for recovery of liver function in patients undergoing hepatectomy and transplantation. Immune reaction of T lymphocytes and macrophages can promote either inflammation or regeneration, immunotolerance or graft rejection.

Applicants have discovered that matrix-embedded endothelial cells (MEECs) rescue dysfunctional endothelium from ischemic liver lobes, restoring blood perfusion and reducing apoptosis. Based on Applicants' experimental observations, MEECs switch the pro-inflammatory profile of Th1 and M1 cells to pro-regenerative Th2 and M2 after hepatectomy. Moreover, MEECs physically bridge injured endothelia of recipient and graft livers and protect from inflammatory reaction and rejection after engraftment. The recovery of endothelium functionality after matrix-embedded endothelial cells implantation improves liver regeneration and hepatocyte function after hepatectomy and engraftment.

Clinically, Applicants' investigation identifies heretofore unappreciated strategies to improve the endothelial and hepatic function of remnant livers after major resection and liver grafts in living donor transplantation. Implantation of MEECs is a solution to the current urgent global need for liver donations—maximizing efficiency of tissue engraftment and recovery, and reducing risks in donors. Implantation of MEECs represents a widely applicable breakthrough in the treatment of ischemia and organ dysfunction in transplantation and provides new techniques for the management of surgery and intervention in urgent care.

The present invention exploits Applicants' discovery that cell-containing implants populated with endothelial cells (MEECs) rescue liver donor and recipient endothelium and parenchyma from ischemic injury after major hepatectomy and engraftment. The inventions disclosed herein highlight the discovery that endothelial-hepatocyte physiologically communicate and cooperate during hepatic repair. The present inventions provide materials and methods for a new approach to improve transplant and regenerative medicine outcomes, for example, liver transplantation.

The benefits of the present inventions are understood by the skilled artisan to extend to most tissues. For example, generally speaking, tissues and organs which are perfused by endothelial cells are suitable for use with the present materials and methods. Similarly, generally speaking tissues and organs which rely on macrophages and T cells for protection from infection are suitable for use. Additionally, generally speaking, tissues which rely on parenchymal cell functionality are suitable for use. Moreover, tissues or organs which are structurally or architecturally similar to the liver such as the kidney are suitable for use with the present materials and methods. Furthermore, tissues or organs which originate from the endoderm as does the liver can benefit from the present inventions, including, digestive tract tissues, stomach tissue, intestinal tissue, pancreatic tissue, bile duct tissues, as well as lung and thyroid. Thus it is expected that the materials and methods of the present invention are suitable for use with tissues selected from the group consisting of: tissues perfused by endothelial cells; tissues which utilize macrophages and T cells; and, tissues typified by parenchymal functionality. Additionally it is expected that the materials and methods of the present invention are suitable for use with tissues selected from the group consisting of: tissues with anatomical architecture like that of liver; and kidney. Furthermore, it is expected that the materials and methods of the present invention are suitable for use with tissues selected from the group consisting of: tissues derived from endoderm; digestive tract tissues, stomach tissue, intestinal tissue, pancreatic tissue, bile duct tissues, lung; and thyroid.

In the case of liver tissue interventions, Applicants' methods and materials mitigate ischemia and maintain tissue integrity under a variety of circumstances when the recipient's standard liver weight is preferably 40% or greater. Applicants' methods and materials are suitable, however, when the recipient's liver weight falls below 40%. This is exemplified by the experiments disclosed herein which demonstrate that 30% can be rescued faster. For purposes of the present invention, the weight of the recipient's tissue to be rescued preferably is about 30% at the time of implantation of MEECs, more preferably about 40%, even more preferably about 50% and most preferably greater than 50% at the time of implantation of MEECs. It is expected that the present invention is suitable for use even when the weight of the recipient's tissue to be rescued is less than 30%, including less than 20% as well as including about 10%. Insofar as the weight of the donor tissue to be implanted contemporaneously with the MEECs, the skilled artisan understands that the total restored tissue volume need not be 100% to benefit from the present invention; and that clinical circumstances and availability of donor tissue dictates the actual volume implanted with the MEECs.

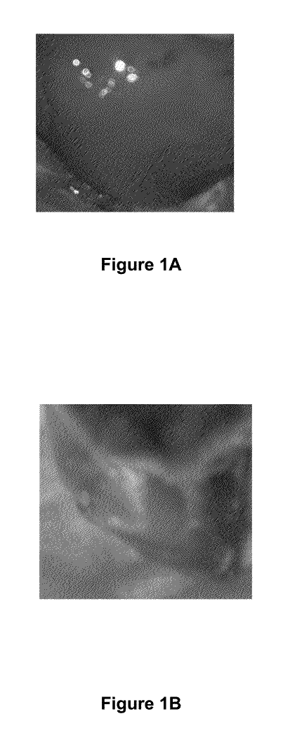

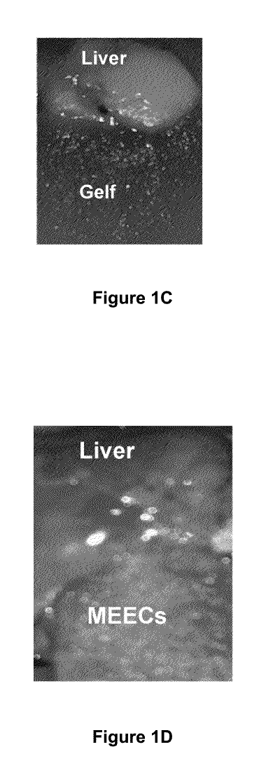

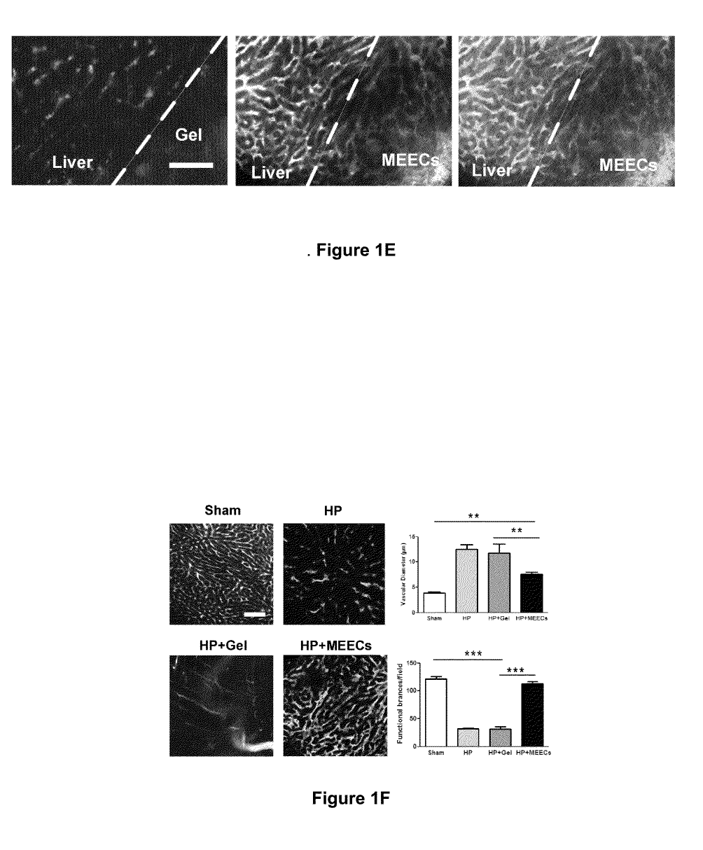

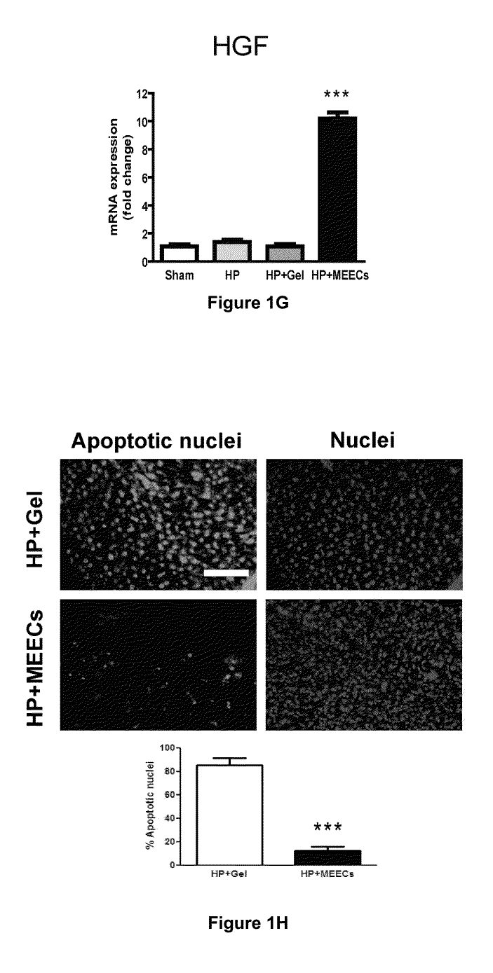

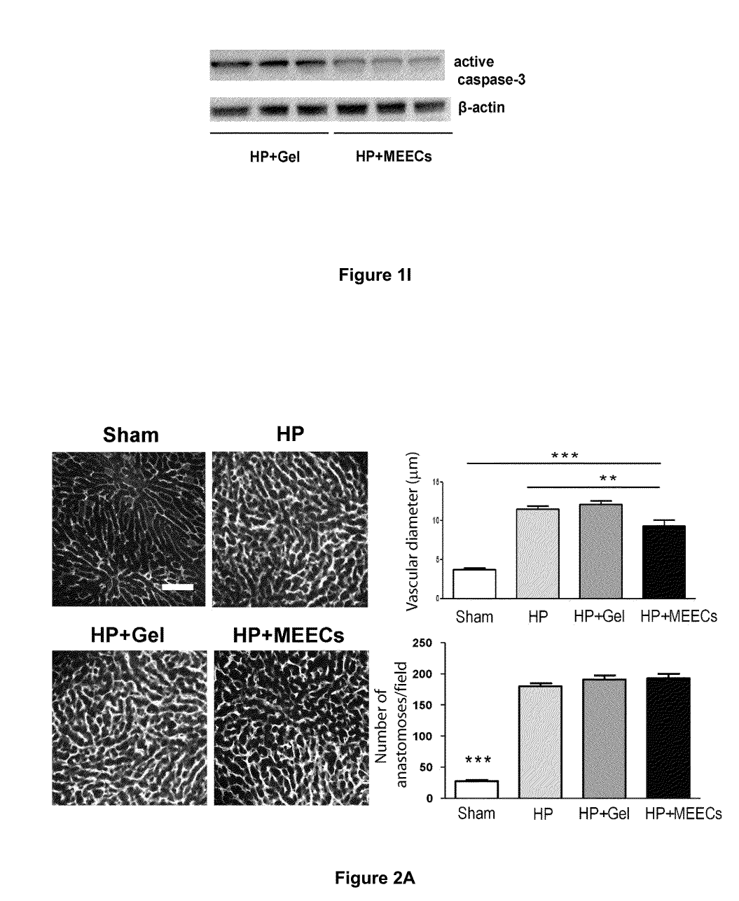

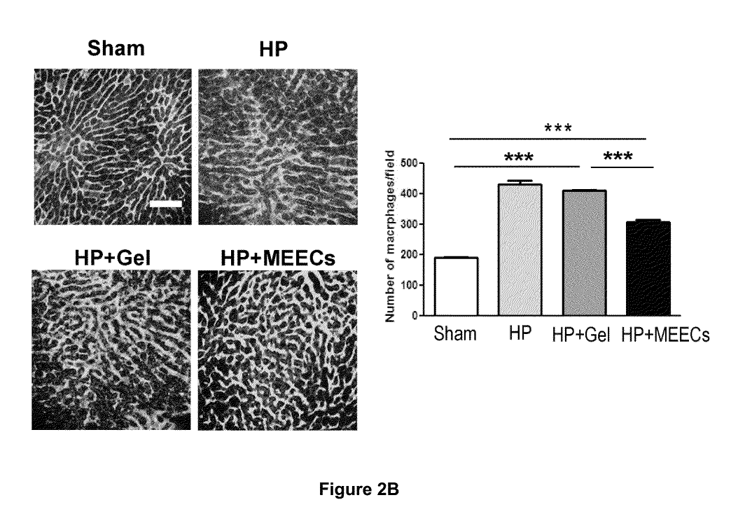

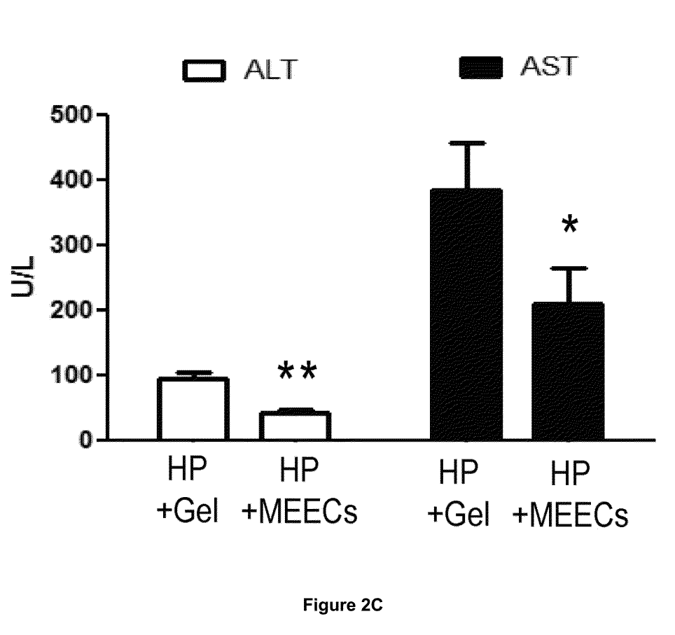

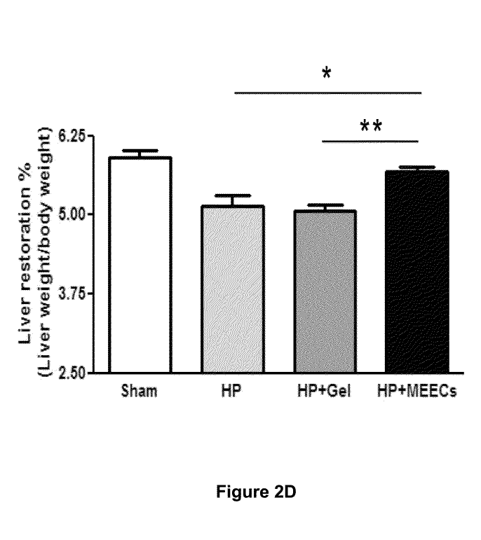

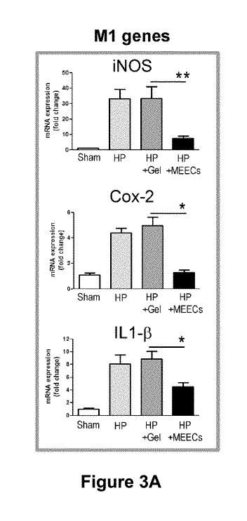

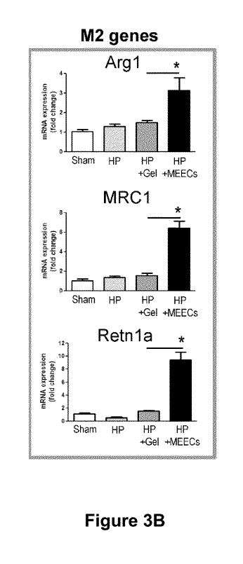

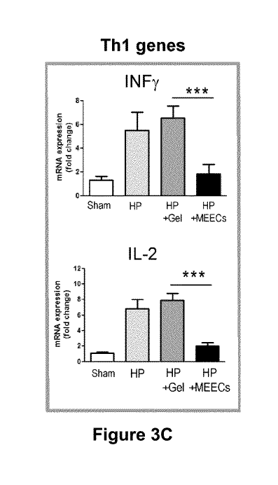

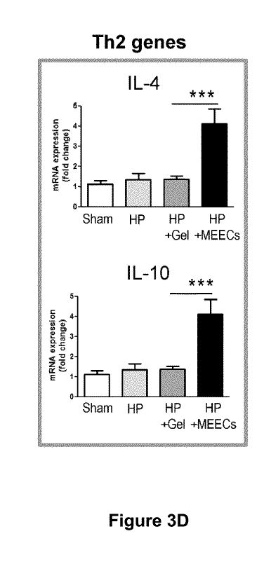

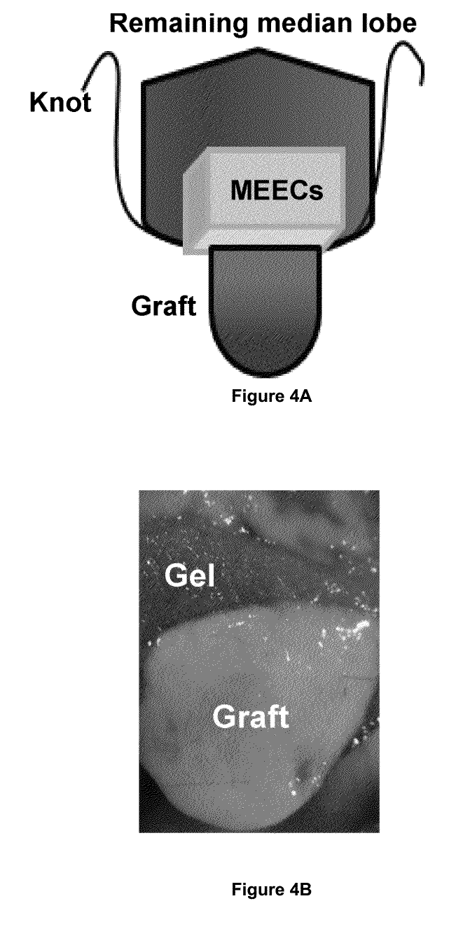

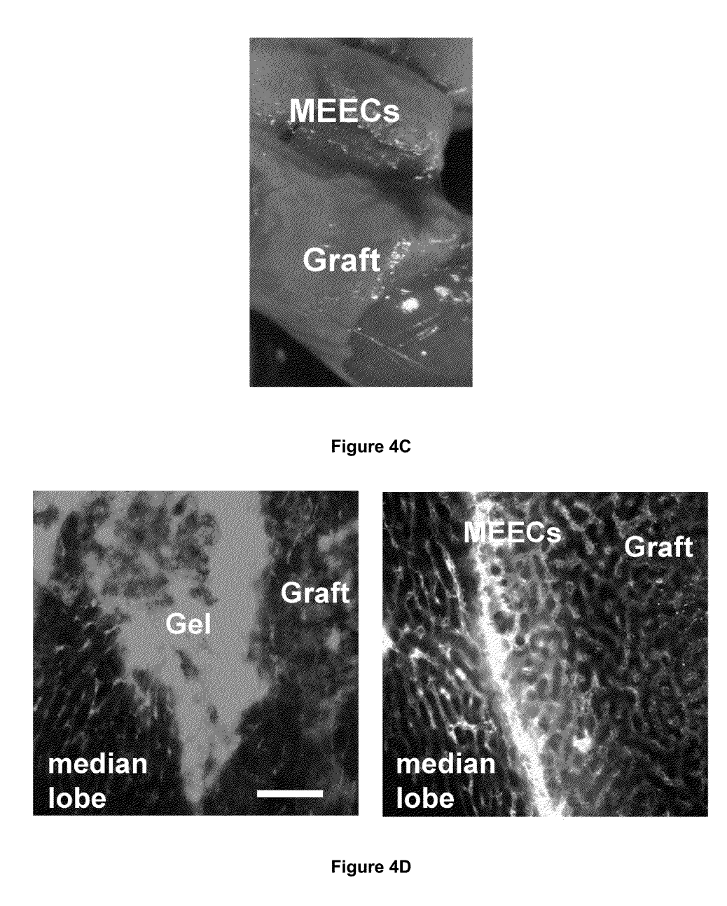

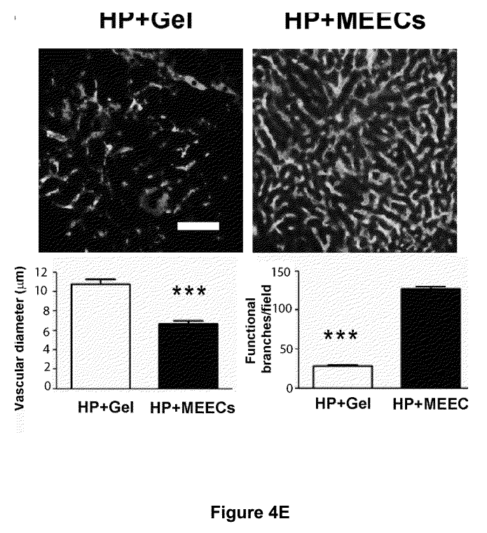

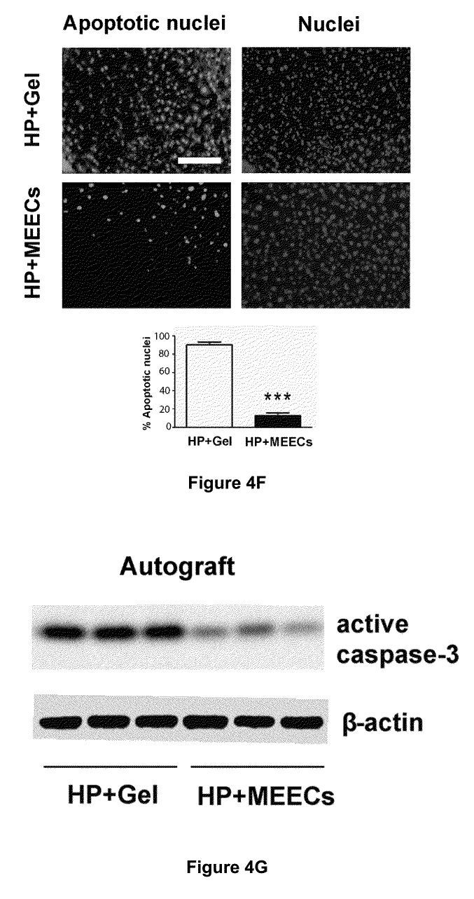

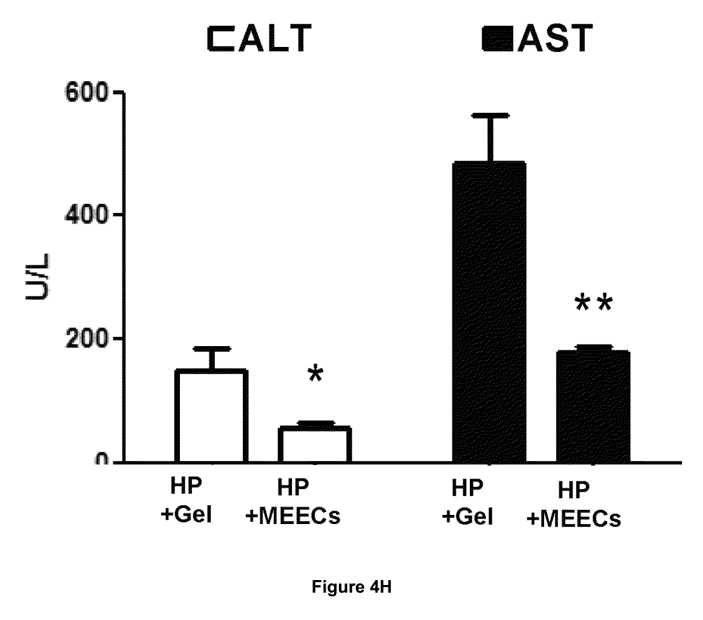

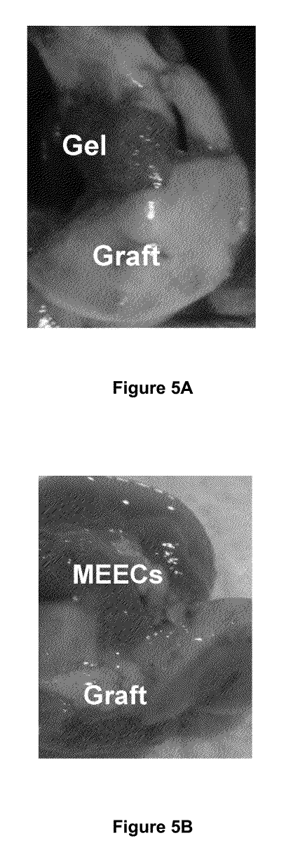

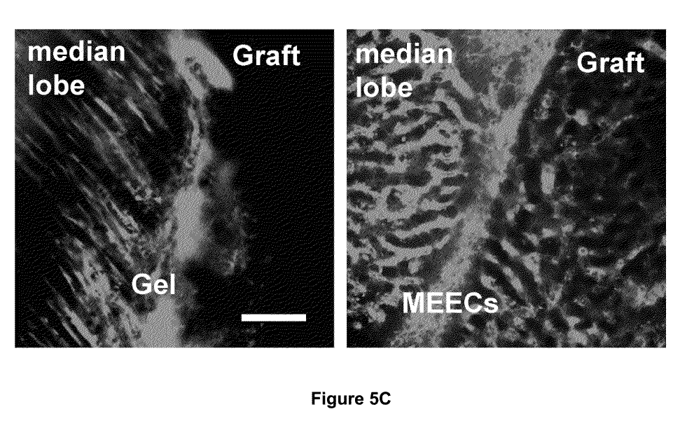

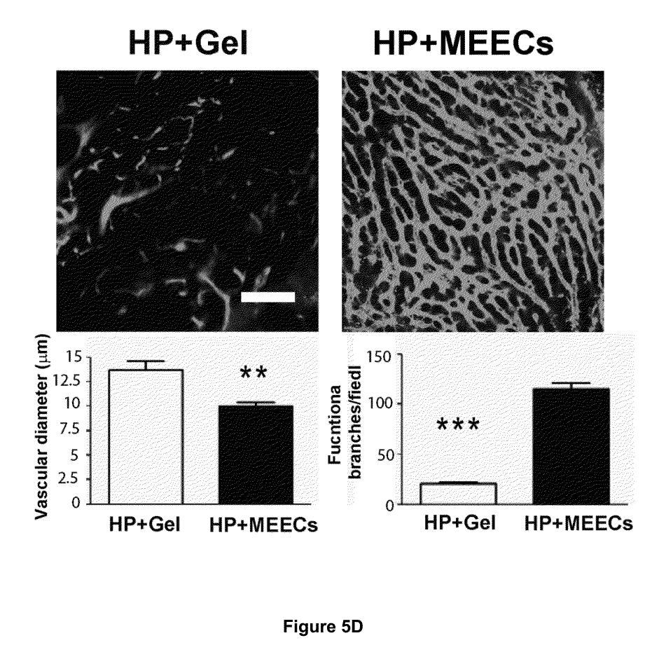

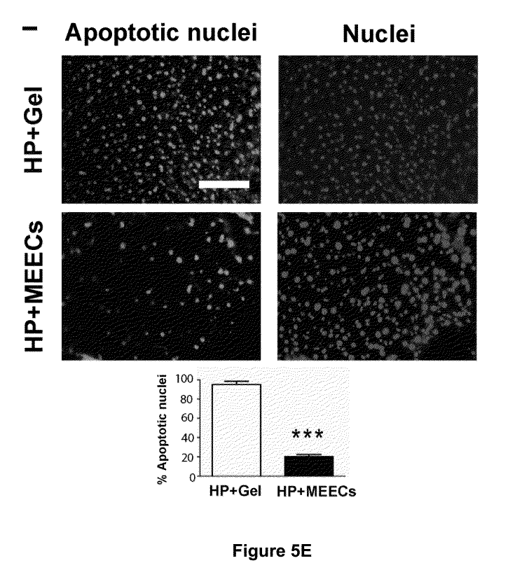

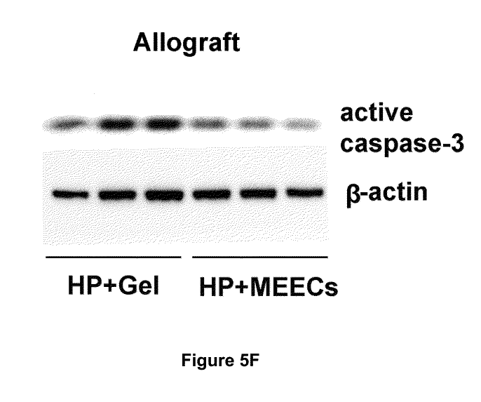

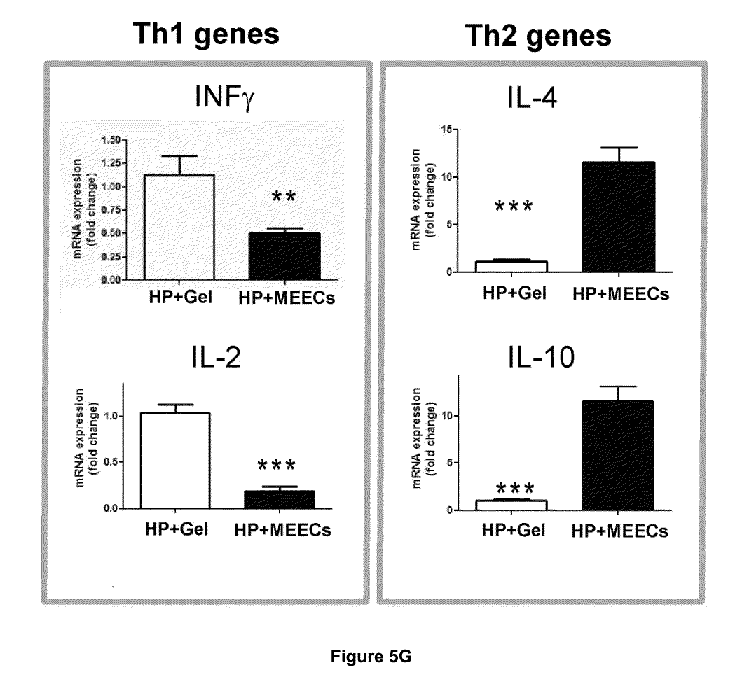

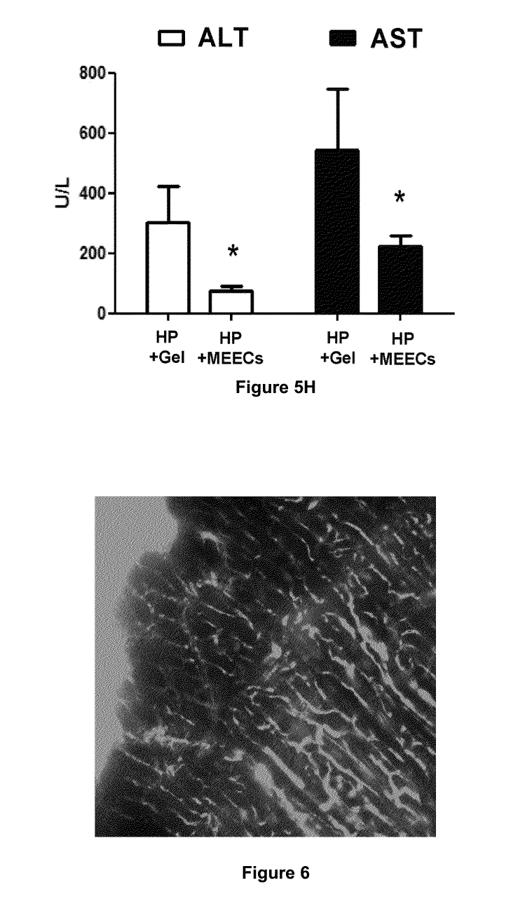

In brief, Applicants have demonstrated unequivocally the beneficial effects of implants of MEECs using a variety of well-recognized parameters in a hepatectomy animal model. With regard to physiological benefits, Applicants have assessed and demonstrated benefits and effects in vascular function. Benefits in vascular function are typified by the findings summarized collectively in FIG. 1 F and FIG. 2A. Similarly, Applicants have assessed and demonstrated benefits and effects on cell survival. Benefits in overcoming and circumventing apoptosis are typified by the findings summarized collectively in FIGS. 1H and 1I. Furthermore, Applicants have assessed and demonstrated benefits and effects in immunomodulation and immunoregulatory function. Such benefits and effects are typified by the findings summarized collectively in FIG. 3. With regard to therapeutic utility, Applicants have unequivocally demonstrated that implants of MEECs facilitate autografts as well as allografts. This is an especially surprising discovery since the implanted MEECs are human-derived and used without clinical complications in murine auto- and allografts. Such findings are summarized collectively in FIGS. 4 and 5, respectively.

Each of the foregoing physiological benefits and therapeutic utilities will now be discussed in more detail:

As illustrated by FIG. 1 and all of its parts, the beneficial effects of MEECs preventing liver damage in ischemic median lobe after 70% hepatectomy are unequivocal. C57BL/6 mice underwent 70% hepatectomy (excision of left lobe and half of median lobe). FIG. 1(A) depicts a macroscopic aspect of a pre-op median lobe; 7 days post-op is depicted in FIG. 1(B) as compared with 7 days post-op with acellular denatured collagen implants (Gel) in FIG. 1(C) versus MEECs as depicted in FIG. 1(D). As depicted in FIG. 1(E), vascularity was analyzed in whole liver by angiography (intracardiac perfusion of FITC-dextran, MW 2×10⁶ Da) using intravital multiphoton microscopy. Macrophages were also stained by intravenous injection of 70 kDa Texas red-dextran 2 hours before angiography and sacrifice. Representative images of the vascular network at the interface between the remaining median lobe and denatured collagen or MEECs are shown in green; macrophages are shown in red and intravascular merge of angiography and Texas red-dextran is shown in yellow. FIG. 1(F) depicts representative images of angiography and quantitative analysis of vascular diameter (congestion) and functional number of vessel branches in the hepatic median lobe of sham or hepatectomized mice (HP) in the presence or absence of acellular implants (HP+Gel) or MEECs (HP+MEECs) 7 days post-op. FIG. 1(G) summarizes gene expression of hepatocyte growth factor (HGF) in ischemic median lobe assessed by Real-time PCR. As seen in FIG. 1(H) to detect cell death, the terminal deoxynucleotidyl transferase dUTP nick-end labeling (TUNEL) assay was used in median liver lobes from hepatectomized mice in contact with acellular implants or MEECs. Representative images of apoptotic nuclei are shown in green. Nuclei were stained with DAPI in blue. Quantification of cell death is shown elsewhere herein. Finally, in FIG. 1(I), a western blot corresponding to active caspase 3 was performed to assess apoptosis in median liver lobes from hepatectomized mice in contact with acellular implants or MEECs. Representative images of three samples of each group to detect active caspase 3 and the housekeeping β-actin are plotted. Scale bars, 100 μm. Data are represented as mean±s.e.m. **P<0.01, ***P<0.001, analysis of variance (ANOVA) or t-student when appropriate.

As illustrated in FIG. 2 and all of its parts, vascular and immunomodulatory effects of MEECs in contact with ischemic median lobe improve liver regeneration and function. C57BL/6 mice underwent 70% hepatectomy (excision of left lobe and half of median lobe). (A) Representative images of angiography and quantitative analysis of vascular diameter (congestion) and angiogenesis (number of anastomoses) in the hepatic right lobe of sham or hepatectomized mice (HP) in the presence or absence of acellular implants (HP+Gel) or MEECs (HP+MEECs) 7 days post-op (B) Representative images and quantitative analysis of total number of macrophages and contacts with vessels in the hepatic right lobe analyzed by injection of 70 kDa Texas red-dextran 2 hours before sacrifice and angiography (intracardiac perfusion of FITC-dextran, MW 2×10⁶ Da) using intravital multiphoton microscopy. Macrophages are shown in red and intravascular merge of angiography and Texas red-dextran is shown in yellow. (C) Serum markers of liver damage Alanine Aminotransferase (ALT) and Aspartate Aminotransferase (AST) were quantified in hepatectomized mice in the presence of acellular implants or MEECs. (D) Liver restoration rate was assessed in sham or hepatectomized mice in the presence or absence of acellular implants or MEECs. Liver restoration rate was calculated as liver weight/body weight×100. Scale bars, 100 μm. Data are represented as mean±s.e.m. *P<0.05, **P<0.01, ***P<0.001, analysis of variance (ANOVA) or t-student when appropriate.

Referring to the entirety of FIG. 3, hepatic immunomodulation of gene expression profiles of macrophages and T helper lymphocytes after implantation of MEECs is evident. Quantification of M1 (iNOS, COX-2 and IL1-β) are depicted in 3(A) and M2 (Arg1, MRC1 and Retn1a) are depicted in 3(B) gene expression profiles by Real-time PCR in sham or hepatectomized mice in the presence or absence of acellular implants (Gel) or MEECs. 3(C) depicts quantification of gene expression profiles of Th1 (INFγ and IL-2) and 3(D) Th2 (IL-4 and IL-10) by Real-time PCR in sham or hepatectomized mice in the presence or absence of acellular implants (Gel) or MEECs. Data are represented as mean of fold change±s.e.m. *P<0.05, **P<0.01, ***P<0.001, analysis of variance (ANOVA).

FIG. 4 underscores Applicants' discovery by depicting the beneficial effects of MEECs in preventing liver damage after autologous engraftment. 4(A) is a schematic representation of surgical implantation of MEECs or acellular implants in the interface between the ischemic median liver lobe and the donated graft from the left liver lobe. 4(B) is a macroscopic aspect of median lobe and autologous grafts implanted with acellular denatured collagen or alternatively as shown in 4(C) MEECs 7 days post-op. 4(D) depicts vascularity determinations made by analyzing the interface between median liver lobe and autologous graft by angiography using intravital multiphoton microscopy. Representative images of the vascular network at the interface between the remaining median lobe, acellular Denatured collagen or MEECs and the graft are shown in green. 4(E) includes representative images of angiography and quantitative analysis of vascular diameter (congestion) and functional number of vessel branches in the hepatic median lobe of hepatectomized mice in the presence of acellular implants (HP+Gel) or MEECs (HP+MEECs) 7 days post-op. 4(F) depicts a means of detect ing intragraft cell death using a TUNEL assay in autologous liver grafts in contact with acellular implants or MEECs. Representative images of apoptotic nuclei are shown in green. Nuclei were stained with DAPI in blue. Quantification of cell death is shown below. 4(G) further assesses apoptosis using Western blot techniques corresponding to active caspase 3 in autologous liver grafts from mice in contact with acellular implants or MEECs. Representative images of three samples of each group to detect active caspase 3 and the housekeeping β-actin are plotted. 4(H) depicts findings relating to serum markers of liver damage Alanine Aminotransferase (ALT) and Aspartate Aminotransferase (AST) by quantification in hepatectomized mice in the presence of acellular implants or MEECs. Scale bars, 100 μm. Data are represented as mean±s.e.m. *P<0.05, **P<0.01, ***P<0.001, analysis of variance (ANOVA) or t-student when appropriate.

FIG. 5 further underscores the beneficial effects of MEECs preventing liver damage after allogeneic engraftment. 5(A) is a macroscopic aspect of median lobe and allogeneic grafts implanted with acellular denatured collagen (Gel) or alternatively as shown in 5(B) MEECs 7 days post-op. 5(C) depicts vascularity assessment in the interface between median liver lobe and allogeneic graft by angiography using intravital multiphoton microscopy. Representative images of the vascular network at the interface between the remaining median lobe, acellular Denatured collagen or MEECs and the graft are shown in green. 5(D) depicts representative images of angiography and quantitative analysis of vascular diameter (congestion) and functional number of vessel branches in the hepatic median lobe of hepatectomized mice in the presence of acellular implants (HP+Gel) or MEECs (HP+MEECs) 7 days post-op. 5(E) illustrates a TUNEL assay performed in allogeneic liver grafts in contact with acellular implants or MEECs to detect cell death. Representative images of apoptotic nuclei are shown in green. Nuclei were stained with DAPI in blue. Quantification of cell death is shown below. 5(F) depicts an assessment of apoptosis using Western blot corresponding to active caspase 3 in allogeneic liver grafts from mice in contact with acellular implants or MEECs. Representative images of three samples of each group to detect active caspase 3 and the housekeeping β-actin are plotted. 5(G) is an intragraft gene expression profile of immunotolerance expressed as Th1 (INFγ and IL-2) and Th2 (IL-4 and IL-10) cytokine expression analyzed by Real-Time PCR. 5(H) depicts serum markers of liver damage Alanine Aminotransferase (ALT) and Aspartate Aminotransferase (AST) as quantified in mice with allografts in the presence of acellular implants or MEECs. Scale bars, 100 μm. Data are represented as mean±s.e.m. *P<0.05, **P<0.01, ***P<0.001, analysis of variance (ANOVA) or t-student when appropriate.

Regarding the outcomes of FIGS. 4 and 5, the data indicate unequivocally that autografts and allografts are possible using the materials and methods of the present invention. In view of the well-known immunomodulatory effects of MEECs referred to elsewhere herein, it is expected that xenografts will benefit from the present invention. Thus the present invention is suitable for use with auto-, allo- and xenografts.

FIG. 6 further underscores Applicant's discovery and its utility. FIG. 6 depicts vascularization of implants of MEECs in contact with an ischemic liver lobe. FIG. 6 depicts the generated functional blood vessels into implants of MEECs as visualized by angiography (intracardiac perfusion of FITC-dextran, MW 2×106 Da) using intravital multiphoton microscopy. Functional blood vessels are shown in green. Magnification is 20×.

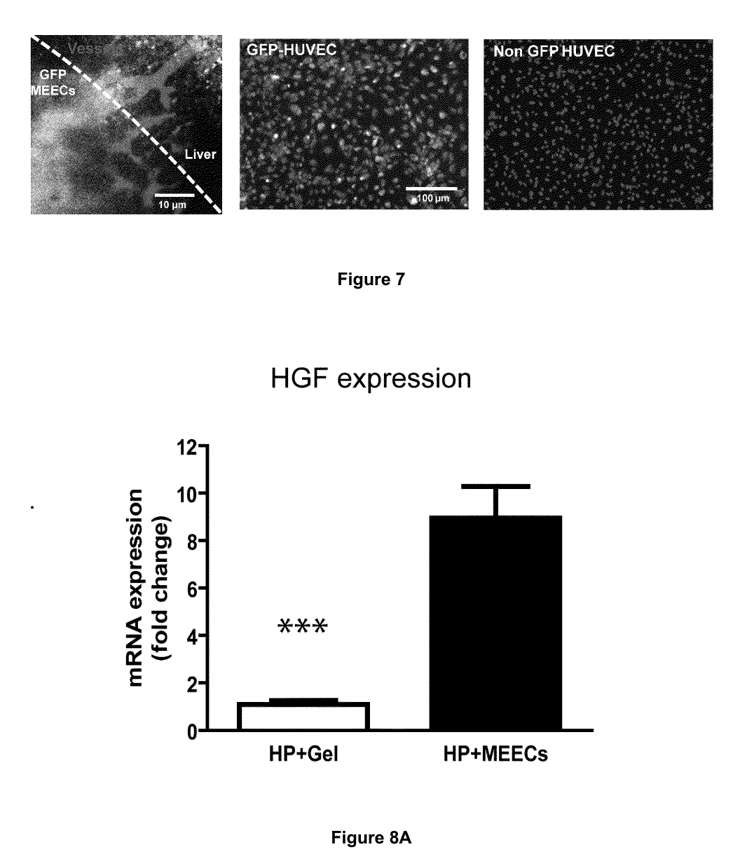

FIG. 7 further depicts the source of angiogenesis into implants of MEECs. As described elsewhere herein, HUVECs constitutively expressing GFP were seeded in gelfoams. Vascularity was analyzed in the interface between implants of HUVECs expressing GFP and implanted median liver lobes of hepatectomized mice by angiography (intracardiac perfusion of Texas red-dextran 70 kda using intravital multiphoton microscopy. A representative image of new vascular anastomoses in the implant interface coming from the extension of hepatic vessels (in red) and from MEEC-generated vessels (in yellow) are shown in the left panel. Control of expression of GFP in HUVEC-GFP cells is shown in the middle panel. Negative control using non-GFP HUVECs is shown in the right panel.

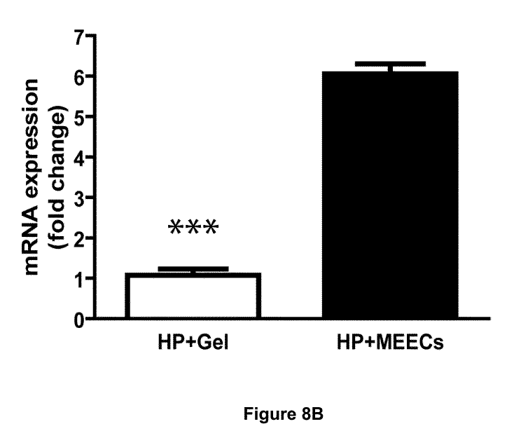

FIG. 8A again summarizes quantification of HGF gene expression profile by Real-time PCR in hepatectomized animals receiving autologous grafts in the presence or absence of acellular implants (Gel) or MEECs while FIG. 8B summarizes quantification of HGF gene expression profile by Real-time PCR in hepatectomized animals receiving allogeneic grafts in the presence or absence of acellular implants (Gel) or MEECs.

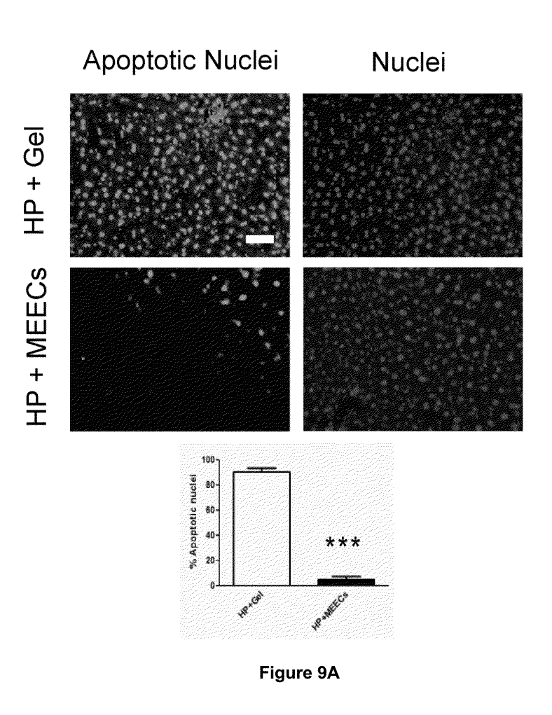

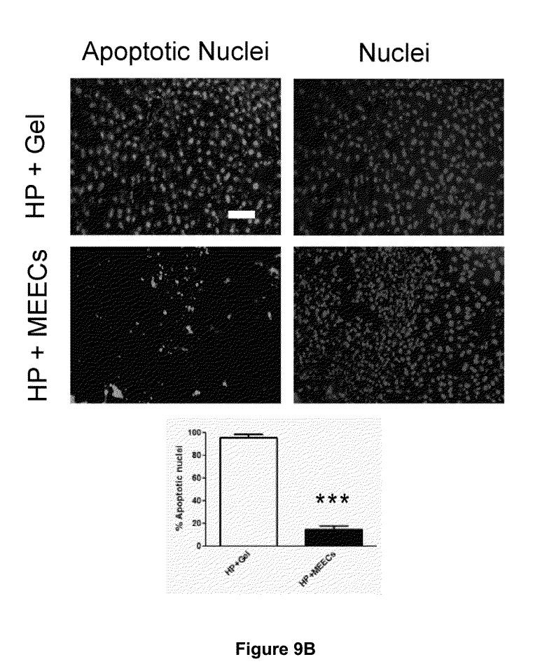

Referring to FIG. 9A, the beneficial effects of MEECs in preventing liver damage in the ischemic median lobe after autologous and allogeneic engraftment are assessed. TUNEL assay was performed to detect cell death in median liver lobe after autologous engraftment in contact with acellular implants or MEECs. Representative images of apoptotic nuclei are shown in green. Nuclei were stained with DAPI in blue. Quantification of cell death is shown elsewhere herein. In a related assessment, FIG. 9B summarizes further the beneficial effects of MEECs preventing liver damage in the ischemic median lobe after autologous and allogeneic engraftment. TUNEL assay was performed in median liver lobe after allogeneic engraftment. Representative images of apoptotic nuclei are shown in green. Nuclei were stained with DAPI in blue. Quantification of cell death is shown elsewhere herein.

### EXAMPLES

### Example 1

### Materials and Methods

### Cell Culture and Seeding of MEECs

MEECs or the implantable material is prepared as described in the following sections:

Human umbilical vein endothelial cells (HUVECs) pooled from 3 donors or HUVECs constitutively expressing GFP were grown in endothelial growth medium supplemented with EGM-2 growth supplements (Lonza). HUVECs (passage 3-5) were first cultured on gelatin-coated tissue culture plates (0.1% gelatin type A, Sigma, St. Louis, Mo.) and then cells were seeded in 3D matrix. For cell-matrix engraftment, compressed denatured collagen matrices (Gelfoam, Pfizer, New York, N.Y.) were cut into 1×1×0.3 cm blocks and hydrated in culture medium at 37° C. for 2 h. Then 4.5×10⁴ ECs (suspended in 50 μL media) were seeded onto one surface of the hydrated matrix and allowed to attach for 1.5 h. Subsequently, the matrix was turned over and additional 4.5×10⁴ ECs were added to infiltrate from the second side. After an additional 1.5 h incubation period to enable cell attachment, each cell-seeded construct was carefully transferred to a separate 30 mL polypropylene tube containing 10 mL of culture medium. Matrices were cultured for 2 weeks, with media changed every 48 h under standard culture conditions (37° C. humidified environment with 5% CO2).

Cell Source

As described herein, the implantable material (also referred to herein as MEECs) of the present invention comprises cells which can be syngeneic, allogeneic, xenogeneic or autologous. In certain embodiments, a source of living cells can be derived from a suitable donor. In certain other embodiments, a source of cells can be derived from a cadaver or from a cell bank. For purposes of the present invention, the cells are non-immortal cells.

In one currently preferred embodiment, cells are endothelial cells. In a particularly preferred embodiment, such endothelial cells are obtained from vascular tissue, preferably but not limited to arterial tissue. As exemplified below, one type of vascular endothelial cell suitable for use is an aortic endothelial cell. Another type of vascular endothelial cell suitable for use is umbilical cord vein endothelial cells. And, another type of vascular endothelial cell suitable for use is coronary artery endothelial cells. Yet other types of vascular endothelial cells suitable for use with the present invention include pulmonary artery endothelial cells and iliac artery endothelial cells.

In another currently preferred embodiment, suitable endothelial cells can be obtained from non-vascular tissue. Non-vascular tissue can be derived from any tubular anatomical structure as described elsewhere herein or can be derived from any non-vascular tissue or organ.

In yet another embodiment, the endothelial cells are endothelial progenitor cells. In another embodiment, the cells can be derived from endothelial progenitor cells or stem cells; in still another embodiment, endothelial cells can be derived from progenitor cells or stem cells generally. In a preferred embodiment, the cells can be progenitor cells or stem cells. In other preferred embodiments, cells can be non-endothelial cells that are syngeneic, allogeneic, xenogeneic or autologous derived from vascular or non-vascular tissue or organ. The present invention also contemplates any of the foregoing which are genetically altered, modified or engineered.

In a further embodiment, two or more types of cells are co-cultured to prepare the present implantable material. For example, a first cell can be introduced into the biocompatible matrix and cultured until confluent. The first cell type can include, for example, smooth muscle cells, fibroblasts, stem cells, endothelial progenitor cells, a combination of smooth muscle cells and fibroblasts, any other desired cell type or a combination of desired cell types suitable to create an environment conducive to endothelial cell growth. Once the first cell type has reached confluence, a second cell type is seeded on top of the first confluent cell type in, on or within the biocompatible matrix and cultured until both the first cell type and second cell type have reached confluence. The second cell type may include, for example, endothelial cells or any other desired cell type or combination of cell types. It is contemplated that the first and second cell types can be introduced step wise, or as a single mixture. It is also contemplated that cell density can be modified to alter the ratio of smooth muscle cells to endothelial cells. Similarly, matrices can be seeded initially with a mixture of different cells suitable for the intended indication or clinical regimen.

All that is required of the anchored and/or embedded cells of the present invention is that they exhibit one or more preferred phenotypes or functional properties. The present invention is based on the discovery that a cell having a readily identifiable phenotype (described elsewhere herein) when associated with a preferred matrix can promote mitigation of ischemia and facilitate tissue resection, regeneration, engraftment and transplantation.

For purposes of the present invention, one such preferred, readily identifiable phenotype typical of cells of the present invention is an altered immunogenic phenotype as measured by the in vitro assays described elsewhere herein. Another readily identifiable phenotype typical of cells of the present invention is an ability to block or interfere with dendritic cell maturation as measured by the in vitro assays described elsewhere herein. This phenotype is referred to herein as an immunomodulatory phenotype.

### Evaluation of One Preferred Phenotype and Immunomodulatory Functionality

For purposes of the invention described herein, the implantable material can be tested for indicia of immunomodulatory functionality prior to implantation. For example, samples of the implantable material are evaluated to ascertain their ability to reduce expression of MHC class II molecules, to reduce expression of co-stimulatory molecules, to inhibit the maturation of co-cultured dendritic cells, and to reduce the proliferation of T cells. In certain preferred embodiments, the implantable material can be used for the purposes described herein when the material is able to reduce expression of MHC class II molecules by at least about 25-80%, preferably 50-80%, most preferably at least about 80%; to reduce expression of co-stimulatory molecules by at least about 25-80%, preferably 50-80%, most preferably at least about 80%; inhibit maturation of co-cultured dendritic cells by at least about 25-95%, preferably 50-95%, most preferably at least about 95%; and/or reduce proliferation of lymphocytes by at least about 25-90%, preferably 50-90%, most preferably at least about 90%.

Levels of expression of MHC class II molecules and co-stimulatory molecules can be quantitated using routine flow cytometry analysis, described in detail below. Proliferation of lymphocytes can be quantitated by in-vitro coculturing 3[H]-thymidine-labeled CD3+-lymphocytes with the implantable composition via scintillation-counting as described below in detail. Inhibition of dendritic cell maturation can be quantitated by either co-culturing the implantable material with dendritic cells and evaluating surface expression of various markers on the dendritic cells by flow cytometry and FACS analysis, or by measuring dendritic cell uptake of FITC-conjugated dextran by flow cytometry. Each of these methods is described in detail below.

In a typical operative embodiment of the present invention, cells need not exhibit more than one of the foregoing phenotypes. In certain embodiments, cells can exhibit more than one of the foregoing phenotypes.

While the foregoing phenotypes each typify a functional endothelial cell, such as but not limited to a vascular endothelial cell, a non-endothelial cell exhibiting such a phenotype(s) is considered endothelial-like for purposes of the present invention and thus suitable for use with the present invention. Cells that are endothelial-like are also referred to herein as functional analogs of endothelial cells; or functional mimics of endothelial cells. Thus, by way of example only, cells suitable for use with the materials and methods disclosed herein also include stem cells or progenitor cells that give rise to endothelial-like cells; cells that are non-endothelial cells in origin yet perform functionally like an endothelial cell using the parameters set forth herein; cells of any origin which are engineered or otherwise modified to have endothelial-like functionality using the parameters set forth herein.

Typically, cells of the present invention exhibit one or more of the aforementioned phenotypes when present in confluent, near-confluent or post-confluent populations and associated with a preferred biocompatible matrix such as those described elsewhere herein. As will be appreciated by one of ordinary skill in the art, confluent, near-confluent or post-confluent populations of cells are identifiable readily by a variety of techniques, the most common and widely-accepted of which is direct microscopic examination. Others include evaluation of cell number per surface area using standard cell counting techniques such as but not limited to a hemocytometer or coulter counter.

Additionally, for purposes of the present invention, endothelial-like cells include but are not limited to cells which emulate or mimic functionally and phenotypically confluent, near-confluent or post-confluent endothelial cells as measured by the parameters set forth herein.

Thus, using the detailed description and guidance set forth below, the practitioner of ordinary skill in the art will appreciate how to make, use, test and identify operative embodiments of the implantable material disclosed herein. That is, the teachings provided herein disclose all that is necessary to make and use the present invention's implantable materials. And further, the teachings provided herein disclose all that is necessary to identify, make and use operatively equivalent cell-containing compositions. At bottom, all that is required is that equivalent cell-containing compositions are effective to modulate an immune response in accordance with the methods disclosed herein. As will be appreciated by the skilled practitioner, equivalent embodiments of the present composition can be identified using only routine experimentation together with the teachings provided herein.

In certain preferred embodiments, endothelial cells used in the implantable material of the present invention are isolated from the aorta of human cadaver donors. Each lot of cells is derived from a single or multiple donors, tested extensively for endothelial cell purity, biological function, the presence of bacteria, fungi, known human pathogens and other adventitious agents. The cells are cryopreserved and banked using well-known techniques for later expansion in culture for subsequent formulation in biocompatible implantable materials. In other embodiments, living cells can be harvested from a donor or from the patient for whom the implantable material is intended.

Cell Preparation

As stated above, suitable cells can be obtained from a variety of tissue types and cell types. In certain preferred embodiments, human aortic endothelial cells used in the implantable material are isolated from the aorta of cadaver donors. In other embodiments, porcine aortic endothelial cells (Cell Applications, San Diego, Calif.) are isolated from normal porcine aorta by a similar procedure used to isolate human aortic endothelial cells. Each lot of cells is derived from a single or multiple donors, tested extensively for endothelial cell viability, purity, biological function, the presence of mycoplasma, bacteria, fungi, yeast, known human pathogens and other adventitious agents. The cells are further expanded, characterized and cryopreserved to form a working cell bank at the third to sixth passage using well-known techniques for later expansion in culture and for subsequent formulation as biocompatible implantable material.

The following is an exemplary protocol for preparing endothelial cells suitable for use with the present invention. Human or porcine aortic endothelial cells are prepared in T-75 flasks pre-treated by the addition of approximately 15 ml of endothelial cell growth media per flask. Human aortic endothelial cells are prepared in Endothelial Growth Media (EGM-2, Cambrex Biosciences, East Rutherford, N.J.). EGM-2 consists of Endothelial Cell Basal Media (EBM-2, Cambrex Biosciences) supplemented with EGM-2 which contain 2% FBS. Porcine cells are prepared in EBM-2 supplemented with 5% FBS and 50 μg/ml gentamicin. The flasks are placed in an incubator maintained at approximately 37° C. and 5% CO2/95% air, 90% humidity for a minimum of 30 minutes. One or two vials of the cells are removed from the −160° C.-140° C. freezer and thawed at approximately 37° C. Each vial of thawed cells is seeded into two T-75 flasks at a density of approximately 3×103 cells per cm3, preferably, but no less than 1.0×103 and no more than 7.0×103; and the flasks containing the cells are returned to the incubator. After about 8-24 hours, the spent media is removed and replaced with fresh media. The media is changed every two to three days, thereafter, until the cells reach approximately 85-100% confluence preferably, but no less than 60% and no more than 100%. When the implantable material is intended for clinical application, only antibiotic-free media is used in the post-thaw culture of human aortic endothelial cells and manufacture of the implantable material of the present invention.

The endothelial cell growth media is then removed, and the monolayer of cells is rinsed with 10 ml of HEPES buffered saline (HEPES). The HEPES is removed, and 2 ml of trypsin is added to detach the cells from the surface of the T-75 flask. Once detachment has occurred, 3 ml of trypsin neutralizing solution (TNS) is added to stop the enzymatic reaction. An additional 5 ml of HEPES is added, and the cells are enumerated using a hemocytometer. The cell suspension is centrifuged and adjusted to a density of, in the case of human cells, approximately 1.75×106 cells/ml using EGM-2 without antibiotics, or in the case of porcine cells, approximately 1.50×106 cells/ml using EBM-2 supplemented with 5% FBS and 50 mg/ml gentamicin.

Biocompatible Matrix

According to the present invention, the implantable material comprises a biocompatible matrix. The matrix is permissive for cell growth, and cell anchoring to and/or embedding within the matrix. A particularly preferred matrix is one characterized by a three-dimensional configuration such that anchored and/or embedded cells can create and occupy a multi-dimensional habitat. Porous matrices are preferred. The matrix can be a solid or a non-solid. Certain non-solid matrices are flowable and suitable for administration via injection-type or infusion-type methods. In certain embodiments, the matrix is flexible and conformable. The matrix also can be in the form of a flexible planar form. The matrix also can be in the form of a gel, a foam, a suspension, a particle, a microcarrier, a microcapsule, or a fibrous structure. In certain preferred embodiments, non-solid forms of matrix to which cells are anchored and/or in which cells are embedded can be injected or infused when administered.

One currently preferred matrix is Gelfoam® (Pfizer, New York, N.Y.), an absorbable gelatin sponge (hereinafter “Gelfoam matrix”). Gelfoam matrix is a porous and flexible sponge-like matrix prepared from a specially treated, purified porcine dermal gelatin solution.

According to another embodiment, the biocompatible matrix material can be a modified matrix material. Modifications to the matrix material can be selected to optimize and/or to control function of the cells, including the cells' phenotype (e.g., the immunomodulatory phenotype) as described elsewhere herein, when the cells are associated with the matrix. According to one embodiment, modifications to the matrix material include coating the matrix with attachment factors or adhesion peptides. Exemplary attachment factors include, for example, fibronectin, fibrin gel, and covalently attached cell adhesion ligands (including for example RGD) utilizing standard aqueous carbodiimide chemistry. Additional cell adhesion ligands include peptides having cell adhesion recognition sequences, including but not limited to: RGDY, REDVY, GRGDF, GPDSGR, GRGDY and REDV.

According to another embodiment, the matrix is a matrix other than Gelfoam. Additional exemplary matrix materials include, for example, fibrin gel, alginate, polystyrene sodium sulfonate microcarriers, collagen coated dextran microcarriers, cellulose, PLA/PGA and pHEMA/MMA copolymers (with polymer ratios ranging from 1-100% for each copolymer). According to a preferred embodiment, these additional matrices are modified to include attachment factors, as recited and described above.

According to another embodiment, the biocompatible matrix material is physically modified to improve cell attachment to the matrix. According to one embodiment, the matrix is cross linked to enhance its mechanical properties and to improve its cell attachment and growth properties. According to a preferred embodiment, an alginate matrix is first cross linked using calcium sulfate followed by a second cross linking step using calcium chloride and routine protocols.

According to yet another embodiment, the pore size of the biocompatible matrix is modified. A currently preferred matrix pore size is about 25 μm to about 100 μm; preferably about 25 μm to 50 μm; more preferably about 50 μm to 75 μm; even more preferably about 75 μm to 100 μm. Other preferred pore sizes include pore sizes below about 25 μm and above about 100 μm. According to one embodiment, the pore size is modified using a salt leaching technique. Sodium chloride is mixed in a solution of the matrix material and a solvent, the solution is poured into a mold, and the solvent is allowed to evaporate. The matrix/salt block is then immersed in water and the salt leached out leaving a porous structure. The solvent is chosen so that the matrix is in the solution but the salt is not. One exemplary solution includes PLA and methylene chloride.

According to an alternative embodiment, carbon dioxide gas bubbles are incorporated into a non-solid form of the matrix and then stabilized with an appropriate surfactant. The gas bubbles are subsequently removed using a vacuum, leaving a porous structure.

According to another embodiment, a freeze-drying technique is employed to control the pore size of the matrix, using the freezing rate of the ice microparticles to form pores of different sizes. For example, a gelatin solution of about 0.1-2% porcine or bovine gelatin can be poured into a mold or dish and pre-frozen at a variety of different temperatures and then lyophilized for a period of time. The material can then be cross-linked by using, preferably, ultraviolet light (254 nm) or by adding glutaraldehyde (formaldehyde). Variations in pre-freezing temperature (for example −20° C., −8° C. or −18° C.), lyophilizing temperature (freeze dry at about −50° C.), and gelatin concentration (0.1% to 2.0%; pore size is generally inversely proportional to the concentration of gelatin in the solution) can all affect the resulting pore size of the matrix material and can be modified to create a preferred material. The skilled artisan will appreciate that a suitable pore size is that which promotes and sustains optimal cell populations having the phenotypes described elsewhere herein.

Cell Seeding of Biocompatible Matrix

The following is a description of one exemplary configuration of a biocompatible matrix. As stated elsewhere, preferred matrices are solid or non-solid, and can be formulated for implantation, injection or infusion.

Pre-cut pieces of a suitable biocompatible matrix or an aliquot of suitable biocompatible flowable matrix are re-hydrated by the addition of EGM-2 without antibiotics at approximately 37° C. and 5% CO2/95% air for 12 to 24 hours. The implantable material is then removed from their re-hydration containers and placed in individual tissue culture dishes. Biocompatible matrix is seeded at a preferred density of approximately 1.5-2.0×105 cells (1.25-1.66×105 cells/cm3 of matrix) and placed in an incubator maintained at approximately 37° C. and 5% CO2/95% air, 90% humidity for 3-4 hours to facilitate cell attachment. The seeded matrix is then placed into individual containers (Evergreen, Los Angeles, Calif.) tubes, each fitted with a cap containing a 0.2 μm filter with EGM-2 and incubated at approximately 37° C. and 5% CO2/95% air. The media is changed every two to three days, thereafter, until the cells have reached confluence. The cells in one preferred embodiment are preferably passage 6, but cells of fewer or more passages can be used.

Cell Growth

A sample of implantable material is removed on or around days 3 or 4, 6 or 7, 9 or 10, and 12 or 13, the cells are counted and assessed for viability, and a growth curve is constructed and evaluated in order to assess the growth characteristics and to determine whether confluence, near-confluence or post-confluence has been achieved. Generally, one of ordinary skill will appreciate the indicia of acceptable cell growth at early, mid- and late time points, such as observation of an exponential increase in cell number at early time points (for example, between about days 2-6 when using porcine aortic endothelial cells), followed by a near confluent phase (for example, between about days 6-8), followed by a plateau in cell number once the cells have reached confluence (for example, between about days 8-10) and maintenance of the cell number when the cells are post-confluent (for example, between about days 10-14).

Cell counts are achieved by complete digestion of the aliquot of implantable material with a solution of 0.5 mg/ml collagenase in a HEPES/Ca++ solution. After measuring the volume of the digested implantable material, a known volume of the cell suspension is diluted with 0.4% trypan blue (4:1 cells to trypan blue) and viability assessed by trypan blue exclusion. Viable, non-viable and total cells are enumerated using a hemocytometer. Growth curves are constructed by plotting the number of viable cells versus the number of days in culture.

For purposes of the present invention, confluence is defined as the presence of at least about 4×10⁵ cells/cm³ when in an exemplary flexible planar form of the implantable material (1.0×4.0×0.3 cm), and preferably about 7×10⁵ to 1×10⁶ total cells per aliquot (50-70 mg) when in an injectable or infusable composition. For both, cell viability is at least about 90% preferably but no less than 80%.

An exemplary embodiment of the present invention comprises a biocompatible matrix and cells suitable for use with any one of the various clinical indications or treatment paradigms described herein. Specifically, in one preferred embodiment, the implantable material comprises a biocompatible matrix and endothelial cells, endothelial-like cells, or analogs of either of the foregoing. In one currently preferred embodiment, the implantable material is in a flexible planar form and comprises endothelial cells, preferably vascular endothelial cells such as but not limited to human aortic endothelial cells and the biocompatible matrix Gelfoam® gelatin sponge (Pfizer, New York, N.Y., hereinafter “Gelfoam matrix”).

Implantable material of the present invention comprises cells anchored to and/or embedded within a biocompatible matrix. Anchored to and/or embedded within means securedly attached via cell to cell and/or cell to matrix interactions such that the cells withstand the rigors of the preparatory manipulations disclosed herein. As explained elsewhere herein, an operative embodiment of implantable material comprises a near-confluent, confluent or post-confluent cell population having a preferred phenotype. It is understood that embodiments of implantable material likely shed cells during preparatory manipulations and/or that certain cells are not as securely attached as are other cells. All that is required is that implantable material comprise cells that meet the functional or phenotypical criteria set forth elsewhere herein.

The implantable material of the present invention was developed on the principals of tissue engineering and represents a novel approach to addressing the herein-described clinical needs. The implantable material of the present invention is unique in that the viable cells anchored to and/or embedded within the biocompatible matrix are able to supply to the site of administration multiple cell-based products in physiological proportions under physiological feed-back control. As described elsewhere herein, the cells suitable for use with the implantable material are endothelial, endothelial-like cells, or analogs of each of the foregoing. Local delivery of multiple compounds by these cells and physiologically-dynamic dosing provide more effective regulation of the processes responsible for modulating an immune response. The implantable material of the present invention can provide an environment which mimics supportive physiology and is conducive to modulation of an immune response.

Evaluation of a Second Preferred Phenotype and Functionality

For purposes of the invention described herein, the implantable material is tested for indicia of functionality prior to delivery to a recipient. For example, as one determination of suitability, conditioned media are collected during the culture period to ascertain levels of heparan sulfate or transforming growth factor-β1 (TGF-β1) or basic fibroblast growth factor (FGF2) or nitric oxide which are produced by the cultured endothelial cells. In certain preferred embodiments, the implantable material can be used for the purposes described herein when total cell number is at least about 1, preferably about 2, more preferably at least about 4×105 cells/cm3 of flexible planar form; percentage of viable cells is at least about 80-90%, preferably ≧90%, most preferably at least about 90%; heparan sulfate in conditioned media is at least about 0.1-0.5 preferably at least about 0.23 microg/mL/day. If other indicia are desired, then TGF-β1 in conditioned media is at least about 200-300, preferably at least about 300 picog/ml/day; FGF2 in conditioned media is below about 200 picog/ml, preferably no more than about 400 picog/ml.

Heparan sulfate levels can be quantitated using a routine dimethylmethylene blue-chondroitinase ABC digestion spectrophotometric assay. Total sulfated glycosaminoglycan (GAG) levels are determined using a dimethylmethylene blue (DMB) dye binding assay in which unknown samples are compared to a standard curve generated using known quantities of purified chondroitin sulfate diluted in collection media. Additional samples of conditioned medium are mixed with chondroitinase ABC to digest chondroitin and dermatan sulfates prior to the addition of the DMB color reagent.

All absorbances are determined at the maximum wavelength absorbance of the DMB dye mixed with the GAG standard, generally around 515-525 nm. The concentration of heparan sulfate per day is calculated by subtracting the concentration of chondroitin and dermatan sulfate from the total sulfated glycosaminoglycan concentration in conditioned medium samples. Chondroitinase ABC activity is confirmed by digesting a sample of purified chondroitin sulfate. Conditioned medium samples are corrected appropriately if less than 100% of the purified chondroitin sulfate is digested. Heparan sulfate levels may also be quantitated using an ELISA assay employing monoclonal antibodies.

If desired, TGF-β1 and FGF2 levels can be quantitated using an ELISA assay employing monoclonal or polyclonal antibodies, preferably polyclonal. Control collection media can also be quantitated using an ELISA assay and the samples corrected appropriately for TGF-β1 and FGF2 levels present in control media. Nitric oxide (NO) levels can be quantitated using a standard Griess Reaction assay. The transient and volatile nature of nitric oxide makes it unsuitable for most detection methods. However, two stable breakdown products of nitric oxide, nitrate (NO3) and nitrite (NO2), can be detected using routine photometric methods. The Griess Reaction assay enzymatically converts nitrate to nitrite in the presence of nitrate reductase. Nitrite is detected colorimetrically as a colored azo dye product, absorbing visible light in the range of about 540 nm. The level of nitric oxide present in the system is determined by converting all nitrate into nitrite, determining the total concentration of nitrite in the unknown samples, and then comparing the resulting concentration of nitrite to a standard curve generated using known quantities of nitrate converted to nitrite.

Also, any one or more of the foregoing assays can be used alone or in combination as a screening assay for identifying a cell as suitable for use with the implantable material of the present invention.

While the earlier-described preferred immunomodulatory phenotype can be assessed using one or more of the optional quantitative heparin sulfate, TGF-β1, NO and/or FGF2 functional assays described above, implantable material can be evaluated for the presence of one or more preferred immunomodulatory phenotypes as follows. For purposes of the present invention, one such preferred, readily identifiable phenotype typical of cells of the present invention is an altered immunogenic phenotype as measured by the in vitro assays described below. Another readily identifiable phenotype typical of cells of the present invention is an ability to block or interfere with dendritic cell maturation as measured by the in vitro assays described below. Each phenotype is referred to herein as an immunomodulatory phenotype and cells exhibiting such a phenotype have immunomodulatory functionality.

Evaluation of Immunomodulatory Phenotype and Functionality

For purposes of the invention described herein, the immunomodulatory functionality of implantable material can be tested as follows. For example, samples of the implantable material are evaluated to ascertain their ability to reduce expression of MHC class II molecules, to reduce expression of co-stimulatory molecules, to inhibit the maturation of co-cultured dendritic cells, and to reduce the proliferation of T cells. In certain preferred embodiments, the implantable material can be used for the purposes described herein when the material is able to reduce expression of MHC class II molecules by at least about 25-80%, preferably 50-80%, most preferably at least about 80%; to reduce expression of co-stimulatory molecules by at least about 25-80%, preferably 50-80%, most preferably at least about 80%; inhibit maturation of co-cultured dendritic cells by at least about 25-95%, preferably 50-95%, most preferably at least about 95%; and/or reduce proliferation of lymphocytes by at least about 25-90%, preferably 50-90%, most preferably at least about 90%.

Levels of expression of MHC class II molecules and co-stimulatory molecules can be quantitated using routine flow cytometry and FACS-analysis, described in detail below. Proliferation of lymphocytes can be quantitated by in-vitro coculturing 3[H]-thymidine-labeled CD3+-lymphocytes with the implantable composition via scintillation-counting as described below in detail. Inhibition of dendritic cell maturation can be quantitated by either co-culturing the implantable material with dendritic cells and evaluating surface expression of various markers on the dendritic cells by flow cytometry and FACS analysis, or by measuring dendritic cell uptake of FITC-conjugated dextran by flow cytometry. Each of these methods is described in detail below.

Also, any one or more of the foregoing assays can be used alone or in combination as a screening assay for identifying a cell as suitable for use with the implantable material of the present invention.

### Animal Model of 70% Hepatectomy and Liver Encraftment

Male C57BL/6 mice were purchased from Charles River Laboratories (Wilmington, Mass. The animals were maintained in a temperature-controlled room (22° C.) on a 12-h light-dark cycle. After arrival, mice were continuously fed ad libitum until euthanasia. Partial hepatectomy was performed as previously described¹. Remaining ischemic median liver lobe was used to attach the implants and the right lobe to assess paracrine effects. For liver engraftment, excised mouse left lobes from a group of ten mice were maintained in warm EGM-2 medium (37° C.) until engraftment to the remaining median lobe of a same or different group of ten mice in the presence or the absence of MEECs or acellular matrices at the interface between recipient and donor liver. Animals were sacrificed after one week. Mouse blood samples were collected by intracardiac puncture. Serum was separated by centrifugation at 3,000×g for 10 min and was then transferred into polypropylene tubes and stored at −80° C. until analysis. Liver restoration rate was calculated as liver weight/body weight×100.

### Whole-Mount Multiphoton Imaging of Macrophage Presence and Angiography in Liver

Mice (9-12 weeks old) were anesthetized with isoflurane. Then they were injected with 100 μL of 20 mg/mL 70,000-kDa Texas red-dextran in Dulbecco's PBS into the tail vein in order to load macrophages by phagocytosis. After 2 hours the animals were euthanized by overexposure to CO₂. Then, mice were perfused transcardially via the left ventricle with phosphate buffered saline (PBS) followed by an injection of fluorescein isothiocyanate-labeled dextran (FITC-dextran, MW 2×10⁶ Da., Sigma, St. Louis, Mo.). Finally, vascular and macrophage fluorescence was visualized under an intravital multiphoton microscope (Leica Microsystems, Heerbrugg, Switzerland). Vascular analysis and macrophage presence was determined by capturing 10 μm z-series of whole liver with a 25×, N.a. 1.05 objective, Olympus FV-1000 MP (Olympus, America Inc, Center Valley, Pa., USA) in which the viewing field is 512×512 μm. Number of macrophages, vascular diameter and anastomosis quantification were analyzed with ImageJ and the tool “angiogenesis analyzer” when appropriate.

### Gene Expression Analysis by Real-Time PCR

Total RNA from liver was extracted using commercially available kits: RNeasy (Gibco-Invitrogen, Paisley, UK). A 1 μg aliquot of total RNA was reverse transcribed using a complementary DNA synthesis kit (High-Capacity cDNA Reverse Transcription Kit, Applied Biosystems, Foster City, Calif., USA). Primers and probes for gene expression assays were selected as follows: M1 profile: NOS2 (Taqman assay reference from Applied Biosystems: Mm00440502_m1), COX2 (Taqman assay reference from Applied Biosystems: Mm00478374_m1), IL-1β (Taqman assay reference from Applied Biosystems: Mm00434228_m1); M2 gene profile: ARG1 (Taqman assay reference from Applied Biosystems: Mm00475988_m1), MRC1 (Taqman assay reference from Applied Biosystems: Mm00485148_m1), RetnIa (Taqman assay reference from Applied Biosystems: Mm00445109_m1); Th1 profile: INFγ (Taqman assay reference from Applied Biosystems: Mm01168134_m1), IL-2 (Taqman assay reference from Applied Biosystems: Mm00434256_m1); Th2 profile: IL-4 (Taqman assay reference from Applied Biosystems Mm00445258_m1), IL-10 (Taqman assay reference from Applied Biosystems: Mm00439614_m1) and hypoxanthine-guanine phosphoribosyltransferase (HPRT), used as an endogenous standard (Taqman assay reference from Applied Biosystems: Mm00446968_m1). Expression assays were designed using the Taqman Gene Expression assay software (Applied Biosystems). Real-time quantitative PCR was analyzed in duplicate and performed with a Lightcycler-480 (Roche Diagnostics). A 10 μl aliquot of the total volume reaction of diluted 1:8 cDNA, Taqman probe and primers and FastStart TaqMan Master (Applied Biosystems) was used in each PCR. The fluorescence signal was captured during each of the 45 cycles (denaturing 10 s at 95 C, annealing 15 s at 60 C and extension 20 s at 72 C). Water was used as a negative control. Relative quantification was calculated using the comparative threshold cycle (CT), which is inversely related to the abundance of mRNA transcripts in the initial sample. The mean CT of duplicate measurements was used to calculate ΔCT as the difference in CT for target and reference. The relative quantity of the product was expressed as fold induction of the target gene compared with the control primers according to the formula 2^{-ΔΔCT}, where ΔΔCT represents ΔCT values normalized with the mean ΔCT of control samples.

### TUNEL Assay

Liver tissue was washed with PBS and fixed with 10% buffered formaldehyde solution for 24 h. Then the tissue was cryo-protected with 30% sucrose solution (in PBS) and then embedded using Tissue-Tek OCT compound (Sakura Fineteck USA, Torrance, Calif.) and frozen. We used the terminal deoxynucleotidyl transferase dUTP nick-end labeling (TUNEL) assay to detect cell death using the fluorescein-FragEL DNA fragmentation detection kit (Calbiochem, San Diego, Calif.) according to the manufacturer's protocol. To quantify and compare the rates of cell death between groups the number of TUNEL-positive cells was counted in relation to the total number of nuclei stained by 4′,6-diamidino-2-phenylindole (DAPI, Vector Laboratories, Burlingame, Calif.). At least eight representative fields were evaluated for each group from which an average value was calculated. Samples were visualized with an epifluorescence microscope (Nikon Eclipse Ti, Kanagawa, Japan).

### Western Blotting

Liver samples were individually homogenized (Polytron PT 1200E, Polytronix Inc, Richardson, Tex.) in RIPA buffer solution (Sigma, St Louis, Mo.) containing 1 mM Na₄P2O₇10H₂O, 20 mM NaF, 1 mM Na₃VO₄ 2 mM and a cocktail of protease inhibitors (Sigma P8340). Phosphorylated VE-cadherin was separated on a SDS-PAGE (10% Novex NuPAGE gel; Life Technologies, Grand Island, N.Y.) and transferred for 7 min to nitrocellulose membranes using the iBlot Gel Transfer Device and iBlot Gel Transfer Stacks (Life Technologies). Thereafter, membranes were blocked (1 h) with 5% powdered defatted milk in TPBS buffer. Then they were incubated overnight with rabbit anti-active caspase-3 polyclonal antibody (1:1000, Abcam, Cambridge, Mass.), followed by incubation with horseradish peroxidase conjugated anti-rabbit antibody (1:1000, Abcam). Bands were visualized by chemiluminescence with Luminata Forte (EMD Millipore).

Unless otherwise stated herein, statistical analyses were as follows: Data are expressed as mean±standard error. Statistical analysis of the results was performed by one-way analysis of variance (ANOVA), the Newman-Keuls test, and the unpaired Student's t test when appropriate. Differences were considered to be significant at a p value of 0.05 or less.

### Example 2

### Animal Model of 70% Hepatectomy and Liver Engraftment

Male C57BL/6 mice (9-12 weeks old) were purchased from Charles River Laboratories (Wilmington, Mass.). The animals were maintained in a temperature-controlled room (22° C.) on a 12 h light-dark cycle. After arrival, mice were continuously fed ad libitum until euthanasia. Partial hepatectomy was performed as previously described.²⁴

Remaining ischemic median liver lobe was used to attach the implants and the right lobe to assess paracrine effects. For liver engraftment, excised mouse left lobes from a group of ten mice were maintained in warm EGM-2 medium (37° C.) until engraftment to the remaining median lobe of a same or different group of ten mice in the presence or the absence of MEECs or acellular matrices at the interface between recipient and donor liver. Animals were sacrificed after one week. Mouse blood samples were collected by intracardiac puncture. Serum was separated by centrifugation at 3,000×g for 10 min and was then transferred into polypropylene tubes and stored at −80° C. until analysis. Liver restoration rate was calculated as liver weight/body weight×100.

Vascularization of implants of MEECs in contact with ischemic liver lobe is depicted in the entirety of FIG. 6. Generated functional blood vessels into implants of MEECs were visualized by angiography (intracardiac perfusion of FITC-dextran, MW 2×106 Da) using intravital multiphoton microscopy. Functional blood vessels are shown in green. Magnification 20×.

### Example 3

### Whole-Mount Multiphoton Imaging of Macrophage Presence and Angiography in Liver, Gene Expression Analysis by Real-Time PCR, TUNEL Assay and Western Blotting

In short, FIG. 7 exemplifies the source of angiogenesis into implants of MEECs. HUVECs constitutively expressing GFP were seeded in gelfoams and grown in endothelial growth medium supplemented with EGM-2 growth supplements for 2 weeks under standard culture conditions (37° C. humidified environment with 5% CO2). Vascularity was analyzed in the interface between implants of HUVECs expressing GFP and implanted median liver lobes of hepatectomized mice by angiography (intracardiac perfusion of Texas red-dextran 70 kda using intravital multiphoton microscopy. Representative image of new vascular anastomoses in the implant interface coming from the extension of hepatic vessels (in red) and from MEEC-generated vessels (in yellow) are shown in the left panel. Control of expression of GFP in HUVEC-GFP cells is shown in the middle panel. Negative control using non-GFP HUVECs is shown in the right panel.

### Example 4

### Experimental Outcomes

1. MEECs Rescue the Ischemic Median Lobe in Mice Undergoing 70% Hepatectomy

Partial hepatectomy (70%) consisted of excising most of healthy median lobe FIG. 1A and the whole left lobe. Acellular matrix or MEECs were implanted adjacent to the remaining ischemic portion of median liver lobe. Seven days later, the animals were sacrificed. At this time the macroscopic aspect of the residual median liver lobe from hepatectomized mice in the absence or the presence of acellular matrix indistinguishably displayed a phenotype of hepatic ischemia with a pale and stiff appearance typical in this animal model (FIG. 1B-C). Only 3 of 10 acellular implants were still attached to the liver at the time of sacrifice. In contrast, all implants with MEECs strongly attached to the median liver lobe one week after implantation and the hepatic tissue macroscopically resembled normal liver (FIG. 1D). As this difference could be explained by a better blood perfusion of median lobes with MEECs, we analyzed the vascular structure at the interface between the injured liver and matrices by angiography. We observed that a new functional vascular network was created into the implant (FIG. 6) that anastomosed host livers (FIG. 1E). This network was not present in acellular matrices of denatured collagen (FIG. 1E). The newly formed vascular anastomoses were originated in part from the extension of hepatic vessels and in part from MEEC-generated angiogenesis as assessed by angiography after implanting ECs constitutively expressing GFP (FIG. 7). A small number of macrophages invaded the implant and were found adjacent to vessel ramifications (FIG. 1E) promoting vascular sprouting as recently reported.²⁵ Vessel bypass between dysfunctional host vessels and implanted MEECs allowed a reduction of blood congestion of the whole median lobe through the significant decrease of the vascular diameter (37%, p<0.01) and preservation of functional vessels (93%, p<0.0001) as compared to acellular matrices or the absence of implant (FIG. 1F). Since MEECs have been reported to attract endothelial progenitor cells (EPC)²⁶ and EPC are responsible of HGF levels after hepatectomy, we quantified hepatic expression of HGF in the ischemic lobe after implantation of MEECs. As expected, gene expression of HGF was not up-regulated in ischemic lobe after hepatectomy or implantation of acellular matrices (FIG. 1G). In contrast, HGF expression was significantly increased when MEECs were implanted (FIG. 1G). To analyze cell damage and apoptosis induced by ischemia we stained median liver lobes using TUNEL assay and analyzed the activation of caspase 3. DNA fragmentation and damage was reduced by 85%, p<0.0001 (FIG. 1H) and apoptosis (i.e. active caspase 3 levels) dropped by 72%, p<0.01 (FIG. 1I) in livers implanted with MEECs as compared with livers receiving acellular implants. Therefore implants of MEECs protect endothelium and parenchyma from death and loss of function in the ischemic lobe of liver donor after hepatectomy.

2. Beneficial Effects of MEECs in Vascular Congestion, Hepatic Function, and Liver Regeneration after Hepatectomy

To analyze the paracrine impact of implantation of MEECs in the regenerating lobes we quantified vascular effects in right lobe 7 days post-op. Livers with or without acellular implant showed an identical increase of vascular diameter in comparison to sham livers (FIG. 2A). In contrast MEEC implants reduced vasodilation without altering angiogenesis in the growing organ expressed as number of new anastomoses (FIG. 2A). The same pattern was observed in the total number of macrophages in the right lobe, that is, an increase of the amount of macrophages after acellular implantation or without matrix and a drop in number of macrophages when MEECs were implanted (FIG. 2B). The recovery of the ischemic lobe by implants of MEECs resulted in an increase of 15% of total liver mass restoration as compared with livers with acellular matrices or without implants (FIG. 2C). This value of liver regeneration using MEECs implies complete recovery of original hepatic mass. As a result of the beneficial effects of MEECs, hepatic injury was reduced as seen in serum levels of ALT and AST (FIG. 2D).

In short, the entirety of FIG. 2 illustrates the beneficial effects of MEECs preventing liver damage in ischemic median lobe after autologous and allogeneic engraftment. In one instance, TUNEL assay was performed to detect cell death in median liver lobe after autologous engraftment in contact with acellular implants or MEECs. Representative images of apoptotic nuclei are shown in green. Nuclei were stained with DAPI in blue. Quantification of cell death is shown below. Tin another instance, TUNEL assay was performed in median liver lobe after allogeneic engraftment. Representative images of apoptotic nuclei are shown in green. Nuclei were stained with DAPI in blue. Quantification of cell death is shown below. Scale bars, 50 μm. Data are represented as mean±s.e.m. ***P<0.001, analysis of variance t-student.

3. MEECs Switch the Phenotype of Macrophages and T-Helper Lymphocytes from Pro-Inflammatory to Anti-Inflammatory and Pro-Regenerative

The reduction of the number of inflammatory cells using MEECs suggested that embedded ECs could have hepatic immunomodulatory effects on macrophage profile stimulating repair and reducing inflammation as reported.²² To identify the phenotype of macrophage subsets in livers after MEECs implantation we quantified the gene expression of M1 (inducible nitric oxide synthase: iNOS; cyclooxygenase 2: COX-2; interleukin 1β: IL1B) and M2 (arginase 1: Arg1; mannose receptor C type 1: MRC1; resistin-like alpha 1: Retn1a) genes by Real-Time PCR. Expression of genes corresponding to the pro-inflammatory macrophage profile M1 was up-regulated in livers without matrix and those receiving acellular matrices—up-regulation that was significantly prevented by implants of MEECs (FIG. 3A). Expression of genes corresponding to the anti-inflammatory and pro-regenerative profile M2 was not significantly up-regulated in livers without MEECs and those receiving acellular matrices but was increased by implants of MEECs (FIG. 3B). It is documented that the switch from M1 to M2 in macrophages is mainly promoted by IL-4 and IL-10 released by Th2 cells²⁷ and that Th2 subset is stimulated in T cells in contact with MEECs²³. We found that hepatic abundance of Th1 genes rose in ischemic lobe after hepatectomy with or without acellular matrix but dropped to physiological levels in livers in contact with MEECs (FIG. 3B). Th2-derived cytokines were only up-regulated when MEECs were implanted (FIG. 3D).

4. MEECs Bridge Vessels from Recipient and Donated Autografts Protecting from Ischemic Injury

Injury derived from ischemia occurs in various clinical settings, such as transplantation, hepatectomy for cancer resection, and hemorrhagic shock¹¹. For that reason, we hypothesized that MEECs could help re-vascularize liver grafts to rescue dysfunctional endothelium in transplantation. We implanted MEECs in the interface between median ischemic lobe after hepatectomy and a liver graft from the left lobe of the same mouse (FIG. 4A). Either median ischemic lobe or autograft displayed a pale color when acellular denatured collagen was implanted (FIG. 4B). In contrast, both remaining median lobe and autograft showed a normal liver color when MEECs were implanted in between (FIG. 4C). Analyzing the vascularity, we found that blood perfusion was very reduced or inexistent in median lobe and autograft in contact with acellular implants. In contrast implanted MEECs bridged vessels between remaining median lobe and autograft (FIG. 4D) and promoted EPC recruitment into the injured lobe as shown by increased levels of HGF (FIG. 8A). Consequently, MEECs preserved vascular functionality in median lobe and reduced vessels diameter and congestion (FIG. 4E). That protection of MEECs against ischemia resulted in a drastic reduction of hepatic median lobe damage (FIG. 9A) and autograft cell injury (85% of reduction) (FIG. 4F) and apoptosis (FIG. 4G). Overall, mice receiving MEECs displayed significantly lower levels of serum transaminases indicating a reduction in hepatocyte damage (FIG. 4H).

5. MEECs Bridge Vessels from Recipient and Donated Allografts Protecting from Ischemic Injury and Immunomodulating a Reduction of Graft Rejection

MEECs attenuate immune rejection in allo- and xenogeneic cell implants.²¹ For this reason, we now analyzed the effects of these implants in hepatic allografts. Median ischemic lobe and allograft displayed a pale color when acellular denatured collagen was implanted and that ischemic color was partially reverted when MEECs were used (FIGS. 5A and B). Vascularity was significantly reduced or entirely obliterated in the median lobe and allograft in contact with acellular implants. In contrast, MEECs connected vessels between the median lobe and allograft (FIG. 5C) and stimulated EPC recruitment into the injured area as shown by enhanced levels of HGF (FIG. 8B). As a result MEECs protected the dysfunctional vascular network in median lobes and reduced congestion (FIG. 5D). These beneficial effects on ischemia were translated into a significant reduction of hepatic median lobe injury (FIG. 9B) and allograft cell death (79% of reduction) (FIG. 5E) and apoptosis (FIG. 5F). Although 50% of immunocompetent mice implanted with allografts died of acute tissue rejection within the first 24 hours, the other half that survived exhibited intragraft immunotolerance expressed as reduction of Th1 (INFγ and IL-2) and increase of Th2 (IL-4 and IL-10) cytokine expression (FIG. 5G). Those mice receiving allografts in the presence of MEECs implants showed improved levels of serum transaminases thus reducing hepatocyte damage (FIG. 5H).

6. Experimental Summary

Ischemic injury is a multifactorial process that affects graft function after liver transplantation. Although recent efforts have improved organ preservation and surgical outcomes²⁸⁻²⁹, there is still a need to understand the basic biology and provide further support of organ viability. Liver ischemia, apoptosis and endothelial dysfunction restrict the success of hepatectomy and liver transplantation. The recovery of blood perfusion in both the recipient and the graft, and protection from adverse inflammatory response are critical events for successful transplantation.² M2 profile of macrophages is potentiated in response to partial hepatectomy or hepatic injury to regenerate the damaged tissue.³⁰ However, M1/M2 balance in macrophages is flexible and the M1 inflammatory phenotype can perpetuate chronic hepatic inflammation and interfere with liver regeneration.³¹ The Examples set forth herein demonstrate that viability of liver sinusoidal endothelium determines the fate of an engrafted hepatic transplants. Implanted matrix-embedded endothelial cells can rescue dysfunctional endothelium in an ischemic liver and stimulate the immune system to boost engraftment and regeneration.

Hepatic sinusoids are lined by a thin layer of functionally unique endothelial cells. LSECs display a high-capacity to clear colloids and soluble waste macromolecules from the circulation to protect hepatocytes, but as such are also the initial target of injury from circulating drugs and toxins and by ischemia-reperfusion injury.² After toxic liver injury, partial hepatectomy or transplantation, damaged LSECs progressively become dysfunctional and may interfere with hepatocyte function and liver regeneration. LSEC progenitor cells arise from the liver and bone marrow (BM LSEC) to contribute to the regenerative response of hepatocytes. These mobilized BM LSEC progenitors engraft in the liver, proliferate and are the highest secretors of the mitogen hepatocyte growth factor (HGF).³²⁻³³ While mature LSECs express and secrete low levels of HGF, high levels of HGF are observed in the liver endothelial progenitor cell and bone marrow-derived LSEC (BM LSEC) progenitors after liver injury.³²⁻³³ LSEC dysfunction or a failure of mobilization of BM LSEC translates into a defective secretion of HGF and an impaired hepatocyte proliferation.³³ MEECs retain high capacity of attracting endothelial progenitors cells.²⁶ The Examples set forth herein show enhanced generation of a functional vascular network that protects ischemic livers and increased hepatic expression of HGF and regeneration. MEECs then boost the mobilization of BM LSEC progenitors to the injured area to stimulate angiogenesis, and this recovery of endothelial cells improves hepatocyte survival, function and liver regeneration. Indeed, the recruitment of BM LSEC is essential for hepatocyte proliferation and restoration of liver mass.³⁴

Controlled inflammation is important to the integration and vascularization of biomaterial scaffolds.³⁵ MEECs achieve an energy state that minimizes stress, shields their immunogenic surface³⁶ and maximizes the secretion of regulatory factors promoting the switch of Th1 to Th2²³ lymphocytes, and the subsequent switch of M1 to M2 macrophages to enhance repair. The Examples set forth herein show interactions of MEECs with injured livers or grafts stimulates the Th2 cytokines IL-4 and IL-10, and the reduction of the Th1 cytokines INFγ and IL-2. IL-4 is required for liver regeneration after partial hepatectomy as IL-4-deficient mice are associated with massive injury, higher morbidity and mortality and impaired liver regeneration.³⁰ The promotion of Th2-derived cytokines in the Th1/th2 balance explains, in part, the faster and total recovery of liver mass after hepatectomy in mice implanted with MEECs. The rescue of dysfunctional endothelium that MEECs promote in the ischemic lobe is an additional contribution to the protection and recovery of liver mass and reduction of apoptosis.

Embedded endothelial cells constructs can be stored for months, and then placed in challenging positions to regulate the local environment. The Examples set forth herein show that when placed in animals that underwent autologous and allogeneic liver grafts MEECs control the local and systemic immune response, promote the bridging between recipient liver and graft vessels and enhance regeneration. These therapeutic angiogenic, immunomodulatory and anti-apoptotic effects of MEECs overcome the current risks of stem cell-derived implants for transplantation as MEECs restore liver function minimizing any concomitant immune reaction. Long-term immunosuppression is required to avoid severe acute and chronic rejection and graft loss in transplanted patients.³⁷ The Examples set forth herein show how in immunocompetent mice MEECs can modulate the behavior of host dysfunctional endothelial cells and immune system to minimize allograft injury and rejection in the absence of any type of immunosuppression. Indeed MEECs reduce the impact of Th1 cytokines and increase Th2 cytokines in mice receiving allografts improving immunotolerance of implants and allografts. Such approach is in line with current strategies aiming to promote stable long-term immunological tolerance of the liver graft.³⁷ MEEC rescuing dysfunctional endothelium and hepatocyte function after hepatectomy is a suitable treatment of ischemia and organ dysfunction in transplantation and provides a pragmatic solution to the urgent global need for liver donations—maximizing efficiency of tissue recovery and reducing risks in donors.

In conclusion, MEECs rescue endothelium function in donor and grafts and also exert immunomodulatory effects to stimulate hepatic repair and regeneration and to reduce liver graft rejection. Since ischemic injury is a common trait in all of transplants and other clinical situations, the present invention provides materials and methods based on the beneficial use of MEECs in liver transplantation and in other ischemia-derived disorders.

### REFERENCES

1. Lim Y S, Kim W R. The global impact of hepatic fibrosis and end-stage liver disease. Clin. Liver Dis. 2008; 12: 733-746.
2. Zhai Y, Petrowsky H, Hong J C, et al. Ischaemia-reperfusion injury in liver transplantation—from bench to bedside. Nat Rev Gastroenterol Hepatol. 2013; 10: 79-89.
3 US Department of Health and Human Services. Organ Procurement and Transplantation Network. 2014. [online], http://optn.transplant.hrsa.gov/data/
4 Takebe T, Sekine K, Enomura M et al. Vascularized and functional human liver from an iPSC-derived organ bud transplant. Nature. 2013; 499: 481-484.
5. Si-Tayeb K, Noto F K, Nagaoka M et al. Highly efficient generation of human hepatocyte-like cells from induced pluripotent stem cells. Hepatology 2010; 51: 297-305.
6. Kulkarni S, Malagò M, Cronin D C. 2nd. Living donor liver transplantation for pediatric and adult recipients. Nat Clin Pract Gastroenterol Hepatol. 2006; 3:149-157.
7. Roll G R, Parekh J R, Parker W F et al. Left hepatectomy versus right hepatectomy for living donor liver transplantation: shifting the risk from the donor to the recipient. Liver Transpl. 2013; 19:472-481.
8. Nissing M H, Hayashi P H. Right hepatic lobe donation adversely affects donor life insurability up to one year after donation. Liver Transpl. 2005; 11: 843-847.
9. Vardanian A J, Busuttil R W, Kupiec-Weglinski J W. Molecular mediators of liver ischemia and reperfusion injury: a brief review. Mol Med. 2008; 14: 337-345.
10. Lentsch A B, Kato A, Yoshidome et al. Inflammatory mechanisms and therapeutic strategies for warm hepatic ischemia/reperfusion injury. Hepatology. 2000; 32:169-173.
11. Eltzschig H K, Eckle T. Ischemia and reperfusion—from mechanism to translation.
Nat Med. 2011; 17: 1391-1401.
12. Tsung A, Klune J R, Zhang X, et al. HMGB1 release induced by liver ischemia involves Toll-like receptor 4 dependent reactive oxygen species production and calcium-mediated signaling. J Exp Med. 2007; 204: 2913-2923.
13. Ding B S, Nolan D J, Butler J M et al. Inductive angiocrine signals from sinusoidal endothelium are required for liver regeneration. Nature 2010; 468: 310-315.
14. Matsumoto K, Yoshitomi H, Rossant J, et al Liver organogenesis promoted by endothelial cells prior to vascular function. Science. 2001; 294: 559-563.
15. DeLeve L D. Liver sinusoidal endothelial cells and liver regeneration. J Clin Invest. 2013; 123(5):1861-66.
16. Sumitran-Holgersson S, Ge X, Karrar A, et al. A novel mechanism of liver allograft rejection facilitated by antibodies to liver sinusoidal endothelial cells. Hepatology. 2004; 40(5):1211-21.
17. Peralta C, Jim\tilde{e}nez-Castro M B, Gracia-Sancho J. Hepatic ischemia and reperfusion injury: effects on the liver sinusoidal milieu. J Hepatol. 2013; 59(5):1094-106.
18. Follenzi A, Benten D, Novikoff P, et al. Transplanted endothelial cells repopulate the liver endothelium and correct the phenotype of hemophilia A mice. J Clin Invest. 2008; 118(3):935-45.
19. Liu F, Liu Z D, Wu N, et al. Transplanted endothelial progenitor cells ameliorate carbon tetrachloride-induced liver cirrhosis in rats. Liver Transpl. 2009; 15(9):1092-100.
20. Methe H, Hess S, Edelman E R. The effect of three-dimensional matrix-embedding of endothelial cells on the humoral and cellular immune response. Semin Immunol. 2008; 20:117-122.
21. Methe H, Groothuis A, Sayegh M H et al. Matrix adherence of endothelial cells attenuates immune reactivity: induction of hyporesponsiveness in allo- and xenogeneic models. FASEB J. 2007; 21: 1515-1526.
22. Methe H, Edelman E R. Cell-matrix contact prevents recognition and damage of endothelial cells in states of heightened immunity. Circulation. 2006; 114: I233-I238.
23. Methe H, Nanasato M, Spognardi A M, et al. T-helper 2 cells are essential for modulation of vascular repair by allogeneic endothelial cells. J Heart Lung Transplant. 2010; 29: 479-486.
24. Willenbring H. A reproducible and well-tolerated method for ⅔ partial hepatectomy in mice. Nat Protoc. 2008; 3: 1167-1170.
24. Mitchell C, Willenbring H. A reproducible and well-tolerated method for ⅔ partial hepatectomy in mice. Nat Protoc. 2008; 3: 1167-1170.
25. Melgar-Lesmes P, Edelman E R. Monocyte-endothelial cell interactions in the regulation of vascular sprouting and liver regeneration in mouse. J Hepatol May 25: 2015. doi: 10.1016/j.jhep.2015.05.011.
26. Indolfi L, laconetti C, Monteforte A, et al. Harnessing Cell: Materials Interactions to Develop Innovative Strategy for the Recruitment of Progenitor Cells. Society for Biomaterials. Annual meeting 2014; Abstract 250
27. Sica A, Invernizzi P, Mantovani A. Macrophage plasticity and polarization in liver homeostasis and pathology. Hepatology. 2014; 59(5):2034-42,
28. Fondevila C, Hessheimer A J, Maathuis M H et al. Hypothermic oxygenated machine perfusion in porcine donation after circulatory determination of death liver transplant. Transplantation 2012; 94: 22-29.
29. Sánchez-Cabus S, Fondevila C, Calatayud D et al. Importance of the temporary portocaval shunt during adult living donor liver transplantation. Liver Transpl. 2013; 19, 174-183.
30. DeAngelis R A, Markiewski M M, Kourtzelis I et al. A complement-IL-4 regulatory circuit controls liver regeneration. J Immunol. 2012; 188(2): 641-8.
31. Xu R, Zhang Z, Wang F S. Liver fibrosis: mechanisms of immune-mediated liver injury. Cell Mol Immunol. 2012; 9(4):296-301.
32. Maher J J. Cell-specific expression of hepatocyte growth factor in liver: Upregulation in sinusoidal endothelial cells after carbon tetrachloride. J Clin Invest 1993; 91(5):2244-52
33. Wang L, Wang X, Xie G, et al. Liver sinusoidal endothelial cell progenitor cells promote liver regeneration in rats. J Clin Invest. 2012; 122(4):1567-73.
34. Harb R, Xie G, Lutzko C, et al. Bone marrow progenitor cells repair rat hepatic sinusoidal endothelial cells after liver injury. Gastroenterology 2009; 137(2):704-12.
35. Spiller K L, Anfang R R, Spiller K J et al. The role of macrophage phenotype in vascularization of tissue engineering scaffolds. Biomaterials. 2014; 35(15):4477-88.
36. Indolfi L, Baker A B, Edelman E R. The role of scaffold microarchitecture in engineering endothelial cell immunomodulation. Biomaterials. 2012: 33(29):7019-27.
37. Adams D H, Sanchez-Fueyo A, Samuel D. From immunosuppression to tolerance. J Hepatol. 2015; 62(1S): S170-S185.

1. A method of treating ischemic tissue comprising the steps of:
contacting a surface of ischemic tissue with a composition comprising
a biocompatible substrate, and
endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and,
incubating the combination of ischemic tissue and composition for a period of time sufficient to reduce or eliminate ischemia in the treated tissue.

2. The method of claim 1 wherein the composition comprises endothelial cells, when contacted with a surface of the ischemic tissue, which are greater than 80% viable and are in a quiescent phase of growth.

3. The method of claim 1 wherein the composition comprises endothelial cells, when contacted with a surface of the ischemic tissue, which express an immunomodulatory phenotype.

4. A method of treating resected tissue comprising the steps of:
contacting a surface of resected tissue with a composition comprising
a biocompatible substrate, and
endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and,
incubating the combination of ischemic tissue and composition for a period of time sufficient to promote viability of the resected tissue.

5. The method of claim 4 wherein the composition comprises endothelial cells, when contacted with a surface of the ischemic tissue, which are greater than 85% viable and are in a quiescent phase of growth.

6. The method of claim 4 wherein the composition comprises endothelial cells, when contacted with a surface of the ischemic tissue, which express an immunomodulatory phenotype.

7. A method of regenerating a tissue comprising the steps of:
contacting a surface of a tissue with a composition comprising
a biocompatible substrate, and
endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and,
incubating the combination of tissue and composition for a period of time sufficient to promote viability and regeneration of the tissue.

8. The method of claim 7 wherein the regenerating tissue is an ischemic tissue, a resected tissue or a transplanted tissue.

9. The method of claim 7 wherein the composition comprises endothelial cells, when contacted with a surface of the ischemic tissue, which are greater than 85% viable and are in a quiescent phase of growth.

10. The method of claim 7 wherein the composition comprises endothelial cells, when contacted with a surface of the ischemic tissue, which express an immunomodulatory phenotype.

11. A method of grafting a donor tissue with a host tissue comprising the steps of:
contacting a surface of a host tissue and a donor tissue with a composition comprising
a biocompatible substrate, and
endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and,
incubating the combination of host tissue, donor tissue and composition for a period of time sufficient to promote formation of a graft comprising host tissue, donor tissue and the composition wherein the composition provides a vascular bridge comprising tubular structures which connect the donor tissue to the host tissue thereby facilitating graft formation.

12. A method of tissue transplantation comprising the steps of:
contacting a surface of a donor tissue with a composition comprising
a biocompatible substrate, and
endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and,
incubating the combination of donor tissue and composition within a transplant recipient for a period of time sufficient to promote viability and integration of the transplanted tissue.

13. A method of forming anastomoses comprising the steps of:
contacting a surface of each of two tissues with a composition comprising
a biocompatible substrate, and
endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and,
incubating the combination of two tissues and composition for a period of time sufficient to promote formation of an anastomoses wherein the composition promotes formation of a vascular bridge comprising tubular structures which connect the tissues thereby facilitating anastomoses formation.

14. A method of inducing de novo formation of vascular structures comprising the steps of:
contacting a surface of a tissue with a composition comprising
a biocompatible substrate, and
endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells; and,
incubating the combination of tissue and composition for a period of time sufficient to promote formation of de novo formation of vasculature within the composition wherein the composition promotes formation of vascularized anatomical structures which are tubular and support blood flow.

15. An implantable composition comprising:
a tissue or segment thereof, and
a cell-containing composition comprising
a biocompatible substrate, and
endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells.

16. The composition of claim 15 wherein the tissue or segment thereof is in contact with the cell-containing composition.

17. A tissue preparation suitable for transplantation comprising:
an organ or a segment thereof, and
a cell-containing composition comprising
a biocompatible substrate, and
endothelial cells adhered to or embedded within the biocompatible substrate (MEECs), wherein the composition has a phenotype characterized by biomarkers selected from the group consisting of heparan sulfate, TGF-beta, FGF2 and nitric oxide and wherein the endothelial cells are non-immortal endothelial cells.

18. The composition of claim 17 wherein the organ or segment thereof is in contact with the cell-containing composition.
